(19) United States
(12) Patent Application Publication
(10) Pub. No.: US 2026/0131008 A1
(43) Pub. Date: May 14, 2026

(54) HYALURONIC ACID BASED DRUG DELIVERY SYSTEMS

(71) Applicant: Cella Therapeutics, LLC, Miami, FL (US)

(72) Inventors: Rhett Schiffman, Laguna Beach, CA (US); Patrick Hughes, Laguna Beach, CA (US)

(21) Appl. No.: 19/439,288

(22) Filed: Jan. 2, 2026

Related U.S. Application Data
(63) Continuation of application No. PCT/US2024/036591, filed on Jul. 2, 2024.
(60) Provisional application No. 63/511,782, filed on Jul. 3, 2023.

(51) Int. Cl. A61K 47/54; A61K 9/00; A61K 31/16; A61K 31/201; A61K 31/216; A61K 45/06; A61P 27/02

(52) U.S. Cl. CPC A61K 47/549; A61K 9/0019; A61K 9/0048; A61K 31/16; A61K 31/201; A61K 31/216; A61K 45/06; A61P 27/02

(57) ABSTRACT

The present disclosure provides a hyaluronic acid-based drug delivery system comprising a compound of Formula (I):

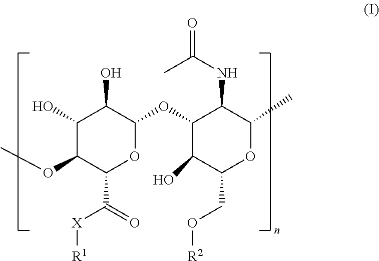

or a pharmaceutically acceptable salt or solvate thereof, wherein X, R¹, and R² are as defined herein. The present disclosure also provides compositions comprising the hyaluronic acid-based drug delivery system described herein and one or more active pharmaceutical ingredient(s). The present disclosure also provides methods of delivering one or more active pharmaceutical ingredient(s) to the eye of a subject in need thereof and methods of treating ophthalmic diseases, disorders, or conditions in a subject in need thereof, comprising administering a composition disclosed herein to the subject.

### CROSS-REFERENCE TO RELATED APPLICATIONS

This application is a continuation application of International Patent Application No. PCT/US2024/036591, filed Jul. 2, 2024, which claims priority to U.S. Provisional Application No. 63/511,782, filed Jul. 3, 2023, all of which are incorporated herein by reference.

### SEQUENCE LISTING

The instant application contains a Sequence Listing which has been submitted electronically in XML format and is hereby incorporated by reference in its entirety. Said XML copy, created on Jul. 1, 2024, is named “4902_029PC01_Seglisting_ST26.xml” and is 32,768 bytes in size.

### FIELD OF THE INVENTION

The present disclosure relates to hyaluronic acid modified by cross-linking, the covalent addition of hydrophobic groups, and/or the covalent addition of one or more active pharmaceutical ingredients. The present disclosure also relates to compositions comprising the hyaluronic acid described herein and one or more active pharmaceutical ingredients. The present disclosure also relates to methods of delivering one or more active pharmaceutical ingredients to one or both eyes of a subject in need thereof and methods of treating ophthalmic diseases, disorders, or conditions in a subject in need thereof, comprising administering a composition disclosed herein to the subject.

### BACKGROUND OF THE INVENTION

Active pharmaceutical ingredients must achieve therapeutic concentrations at the target tissue for the appropriate duration to have a beneficial effect. This can be extremely challenging for ocular drugs, as not every active pharmaceutical ingredient can be formulated topically. For those active pharmaceutical ingredients that can be formulated topically, the bioavailability of those in the aqueous humor is usually only 1 to 5 percent.

Injectable formulations can potentially be provided by in situ gelling depots and hydrogels or sustained delivery particulates sequestered by in situ gelling depots or hydrogels. Injectable depots are attractive for delivery to the posterior segment of the eye because they can potentially deliver a high payload through a small gauge needle. The materials are often hydrophilic and hydrated, conferring a degree of biocompatibility.

Hyaluronic acid (HA) and collagen (Col) are attractive polymers for the design of intraocular drug delivery systems as both are naturally present in the vitreous. HA has a history of clinical use as a viscosupplement or surgical aid with an extensive positive safety profile. Non-crosslinked HA is known to clear from the posterior segment quickly relative to a desired 3 to 12 month, or longer, delivery profile.

Therefore, there remains a need to develop drug delivery systems and compositions thereof that are capable of sustained release of one or more active pharmaceutical ingredient(s) over a controlled time period.

### BRIEF SUMMARY OF THE INVENTION

The present disclosure provides hyaluronic acid comprising a compound of Formula (I):

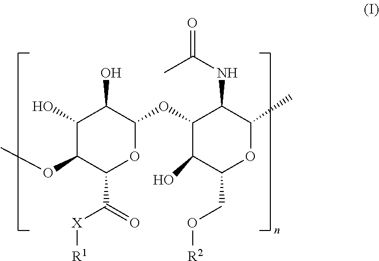

or a salt or solvate thereof, wherein:
n is an integer from 500 to 25,000;
X at each occurrence is selected from the group consisting of —NH— and —O—;
R¹ and R² at each occurrence are independently selected from the group consisting of hydrogen, a hydrophobic group, -L¹-L²-A, and -A;
each L¹ is an amino acid or absent;
each L² is a linker; and
each A is independently a covalently bonded active pharmaceutical ingredient, wherein each instance of -A can be the same or different.

In some aspects, n is from 1,200 to 5,000.

In some aspects, X is —NH—.

In some aspects, X is —O—.

In some aspects, the hydrophobic group is selected from the group consisting of optionally substituted C₁-C₁₈ alkyl and an amino acid.

In some aspects, the hydrophobic group is C₁-C₁₈ alkyl that is substituted with a phenyl group.

In some aspects, the hydrophobic group is a C₁-C₁₈ alkyl that is selected from the group consisting of:

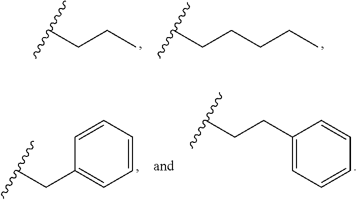

In some aspects, the hydrophobic group is an amino acid that is selected from the group consisting of:

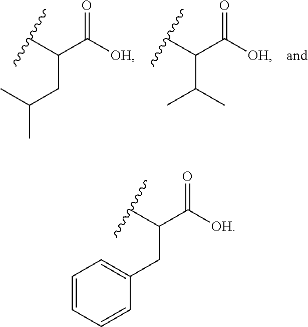

In some aspects, L¹ is selected from the group consisting of:

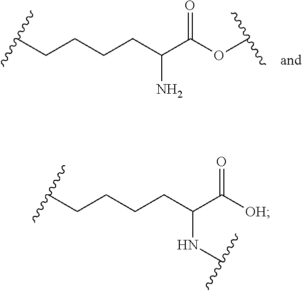 [SMILES: *CCCCC(N)C(=O)O* ; *CCCCC(N*)C(=O)O]

In some aspects, L² is selected from the group consisting of:

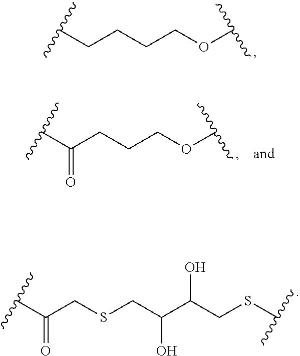 [SMILES: *CCCCO* ; *OCCCC(*)=O ; *SCC(O)C(O)CSCC(*)=O]

In some aspects, each A is independently selected from the group consisting of an intraocular pressure (IOP) lowering agent, a CNTF analog, a FAS inhibitor or FAS ligand inhibitor, a HIF-1 alpha inhibitor, a HIF-2 alpha inhibitor, a VEGF inhibitor, a VIP analog, a histatin analog, an alpha crystallin aggregation inhibitor, a reducing agent, a mitochondrial stabilizer, and a TNF-alpha inhibitor.

In some aspects, the IOP lowering agent is a prostaglandin, a beta blocker, an alpha agonist, a carbonic anhydrase inhibitor, a cholinergic agent, a rho kinase inhibitor, or a cannabinoid receptor agonist.

In some aspects, the prostaglandin is a prostaglandin compound, a prostaglandin EP2 agonist, a prostaglandin EP3 agonist, a nitric oxide donating prostaglandin compound, or a combination thereof.

In some aspects, the prostaglandin compound is bimatoprost, bimatoprost acid, travoprost, travoprost acid, latanoprost, latanoprost acid, latanoprostene, tafluprost, or tafluprost acid.

In some aspects, the prostaglandin EP2 agonist is taprenepag or omidenepag isopropyl.

In some aspects, the prostaglandin EP3 agonist is dinoprostone, misoprostol, limaprost, gemeprost, alprostadil, or rivenprost.

In some aspects, the nitric oxide donating prostaglandin compound is latanoprostene bunod.

In some aspects, the beta blocker is timolol, betaxolol, levobunolol, or metipranolol.

In some aspects, the alpha agonist is brimonidine or apraclonidine.

In some aspects, the carbonic anhydrase inhibitor is brinzolamide, acetazolamide, dorzolamide, or methazolamide.

In some aspects, the cholinergic agent is pilocarpine or carbachol.

In some aspects, the rho kinase inhibitor is netarsudil.

In some aspects, the CNTF analog is SEQ ID NO: 18, SEQ ID NO.: 1, SEQ ID NO: 2, or recombinant CNTF.

In some aspects, the FAS inhibitor is bicyclol, FLIP, MET12 (SEQ ID NO: 3), compound 1 (SEQ ID NO: 5), compound 2 (SEQ ID NO: 6), compound 3 (SEQ ID NO: 7), compound 4 (SEQ ID NO: 8), compound 5 (SEQ ID NO: 9), compound 6 (SEQ ID NO: 10), compound 7 (SEQ ID NO: 11), compound 8 (SEQ ID NO: 12), compound 9 (SEQ ID NO: 13), compound 10 (SEQ ID NO: 14), compound 11 (SEQ ID NO: 15), ONL1204, SEQ ID NO: 4, FAIM, NOL3, human decoy receptor 1 (DcRl), human decoy receptor 2 (DcR2), or human decoy receptor 3 (DcR3).

In some aspects, the TNF-alpha inhibitor is etanercept, infliximab, golimumab, certolizumab, adalimumab, TNFRI-selective antagonistic mutant TNF (RlantTNF), DMS5540, TNF Receptor-One Silencer (TROS), or ATROSAB.

In some aspects, the cannabinoid receptor agonists is Δ⁹-tetrahydrocannabinol (Δ⁹-THC), anandamide, 2-arachidonoyl glycerol, 1,1-dimethylheptyl-11-hydroxy-tetrahydrocannabinol (HU-210), (−)-cis-3-[2-hydroxy-4-(1,1-dimethylheptyl)phenyl]-trans-4-(3-hydroxypropyl)cyclohexanol (CP55940), (R)-(+)-[2,3-dihydro-5-methyl-3-(4-morpholinylmethyl)pyrrolo-[1,2,3-de]-1,4-benzoxazin-6-yl]-1-naphthalenylmethanone (R-(+)-WIN55212), (2-methyl-1-propyl-1H-indol-3-yl)-1-naphthalenylmethanone (JWH-015), 3-(1,1-dimethylbutyl)-6,6,9-trimethyl-6a,7,10,10a-tetrahydro-6H-benzo[c]chromene (JWH-133), onternabez (HU-308), 1-(methylpiperidin-2-ylmethyl)-3-(2-iodo-5-nitrobenzoyl)indole (AM1241), 1-(2,3-Dichlorobenzoyl)-5-methoxy-2-methyl-3-[2-(4-morpholinyl)ethyl]-1H-indole (GW405833), 2-[(2,4-dichlorophenyl)amino]-N-[(tetrahydro-2H-pyran-4-yl)methyl]-4-(trifluoromethyl)-5-pyrimidinecarboxamide (GW842166X), or 1-(4-(1,1-dimethylheptyl)-2,6-dimethoxyphenyl)-3-methylcyclohexanol (O-1966).

In some aspects, from about 1% to about 50% of the R groups are hydrophobic groups.

In some aspects, from about 1% to about 50% of the R groups are -L¹-L²-A or -A.

In some aspects, each -A of Formula (I) is selected from one active pharmaceutical ingredient.

In some aspects, each -A of Formula (I) is selected from two active pharmaceutical ingredients.

In some aspects, each -A of Formula (I) is selected from three active pharmaceutical ingredients.

In some aspects, the compound of Formula (I) is prepared by:
(i) reacting unsubstituted hyaluronic acid with a first active pharmaceutical ingredient; and
(ii) reacting the product of step (i) with a second active pharmaceutical ingredient;
to provide the hyaluronic acid.

In some aspects, the compound of Formula (I) is prepared by:
(i) reacting unsubstituted hyaluronic acid with a first active pharmaceutical ingredient;
(ii) reacting the product of step (i) with a second active pharmaceutical ingredient; and
(iii) reacting the product of step (ii) with a third active pharmaceutical ingredient;
to provide the hyaluronic acid.

In some aspects, the hyaluronic acid comprises a first compound of Formula (I) that is cross-linked with a second compound of Formula (I), wherein the first compound comprises a first active pharmaceutical ingredient and the second compound comprises a second active pharmaceutical ingredient.

In some aspects, the hyaluronic acid comprises a first compound of Formula (I) that is cross-linked with a second compound of Formula (I) and a third compound of Formula (I), wherein the first compound comprises a first active pharmaceutical ingredient, the second compound comprises a second active pharmaceutical ingredient, and the third compound comprises a third active pharmaceutical ingredient.

The present disclosure also provides a composition comprising the hyaluronic acid disclosed herein and a free active pharmaceutical ingredient.

In some aspects, the free active pharmaceutical ingredient is selected from the group consisting of an IOP lowering agent, a CNTF analog, a FAS inhibitor, a HIF-1 alpha inhibitor, a HIF-2 alpha inhibitor, a VEGF inhibitor, a VIP analog, a histatin analog, an alpha crystallin aggregation inhibitor, a reducing agent, and a TNF-alpha inhibitor.

In some aspects, the IOP lowering agent is a prostaglandin, a beta blocker, an alpha agonist, a carbonic anhydrase inhibitor, a cholinergic agent, a rho kinase inhibitor, or a cannabinoid receptor agonist.

In some aspects, the prostaglandin is a prostaglandin compound, a prostaglandin EP2 agonist, a prostaglandin EP3 agonist, a nitric oxide donating prostaglandin compound, or a combination thereof.

In some aspects, the prostaglandin compound is bimatoprost, bimatoprost acid, travoprost, travoprost acid, latanoprost, latanoprost acid, latanoprostene, tafluprost, or tafluprost acid.

In some aspects, the prostaglandin EP2 agonist is taprenepag or omidenepag isopropyl.

In some aspects, the prostaglandin EP3 agonist is dinoprostone, misoprostol, limaprost, gemeprost, alprostadil, or rivenprost.

In some aspects, the nitric oxide donating prostaglandin compound is latanoprostene bunod.

In some aspects, the beta blocker is timolol, betaxolol, levobunolol, or metipranolol.

In some aspects, the alpha agonist is brimonidine or apraclonidine.

In some aspects, the carbonic anhydrase inhibitor is brinzolamide, acetazolamide, dorzolamide, or methazolamide.

In some aspects, the cholinergic agent is pilocarpine or carbachol.

In some aspects, the rho kinase inhibitor is netarsudil.

In some aspects, the CNTF analog is SEQ ID NO: 18, SEQ ID NO.: 1, SEQ ID NO: 2, or recombinant CNTF.

In some aspects, the FAS inhibitor is bicyclol, FLIP, MET12 (SEQ ID NO: 3), compound 1 (SEQ ID NO: 5), compound 2 (SEQ ID NO: 6), compound 3 (SEQ ID NO: 7), compound 4 (SEQ ID NO: 8), compound 5 (SEQ ID NO: 9), compound 6 (SEQ ID NO: 10), compound 7 (SEQ ID NO: 11), compound 8 (SEQ ID NO: 12), compound 9 (SEQ ID NO: 13), compound 10 (SEQ ID NO: 14), compound 11 (SEQ ID NO: 15), ONL1204, SEQ ID NO: 4, FAIM, NOL3, human decoy receptor 1 (DcRl), human decoy receptor 2 (DcR2), or human decoy receptor 3 (DcR3).

In some aspects, the TNF-alpha inhibitor is etanercept, infliximab, golimumab, certolizumab, adalimumab, TNFRl-selective antagonistic mutant TNF (RlantTNF), DMS5540, TNF Receptor-One Silencer (TROS), or ATROSAB.

In some aspects, the cannabinoid receptor agonists is Δ⁹-tetrahydrocannabinol (Δ⁹-THC), anandamide, 2-arachidonoyl glycerol, 1,1-dimethylheptyl-11-hydroxy-tetrahydrocannabinol (HU-210), (−)-cis-3-[2-hydroxy-4-(1,1-dimethylheptyl)phenyl]-trans-4-(3-hydroxypropyl)cyclohexanol (CP55940), (R)-(+)-[2,3-dihydro-5-methyl-3-(4-morpholinylmethyl)pyrrolo-[1,2,3-de]-1,4-benzoxazin-6-yl]-1-naphthalenylmethanone (R—(+)-WIN55212), (2-methyl-1-propyl-1H-indol-3-yl)-1-naphthalenylmethanone (JWH-015), 3-(1,1-dimethylbutyl)-6,6,9-trimethyl-6a,7,10,10a-tetrahydro-6H-benzo[c]chromene (JWH-133), onternabez (HU-308), 1-(methylpiperidin-2-ylmethyl)-3-(2-iodo-5-nitrobenzoyl)indole (AM1241), 1-(2,3-Dichlorobenzoyl)-5-methoxy-2-methyl-3-[2-(4-morpholinyl)ethyl]-1H-indole (GW405833), 2-[(2,4-dichlorophenyl)amino]-N-[(tetrahydro-2H-pyran-4-yl)methyl]-4-(trifluoromethyl)-5-pyrimidinecarboxamide (GW842166X), or 1-(4-(1,1-dimethylheptyl)-2,6-dimethoxyphenyl)-3-methylcyclohexanol (O-1966).

In some aspects, the concentration of the free active pharmaceutical ingredient is from about 1 wt % to about 20 wt %.

In some aspects, the concentration of the hyaluronic acid is from about 0.1 wt % to about 5 wt %.

In some aspects, the composition further comprises a second polymer.

In some aspects, the second polymer is a collagen or a poloxamer.

In some aspects, the poloxamer is Pluronic® L64, Pluronic® P65, Pluronic® P84, Pluronic® P85, Pluronic® F88, Pluronic® P103, Pluronic® P104, Pluronic® P105, Pluronic® F108, Pluronic® F123, or Pluronic® F127.

In some aspects, the concentration of the hyaluronic acid is from about 1 mg/mL to about 10 mg/mL.

In some aspects, the concentration of the second polymer is from about 1 mg/mL to about 250 mg/mL.

In some aspects, the concentration of the second polymer is from about 1 mg/mL to about 5 mg/mL.

In some aspects, the bound active pharmaceutical ingredient is released over a period of from about 6 months to about 24 months.

In some aspects, the free active pharmaceutical ingredient is released over a period of from about 6 months to about 24 months.

In some aspects, the hyaluronic acid is a hydrogel.

In some aspects, the composition is a solid implant.

The present disclosure also provides a method of delivering an active pharmaceutical ingredient to the anterior chamber, posterior chamber, suprachoroidal space, or vitreous humor of one or both eyes of a subject in need thereof, the method comprising injecting a hyaluronic acid disclosed herein or a composition disclosed herein into one or both eyes of the subject, or implanting a composition disclosed herein into one or both eyes of the subject.

The present disclosure also provides a method of treating glaucoma, age-related macular degeneration (AMD), macular degeneration (MD), uveitis, retinal vein occlusion, macular edema, macular telangiectasia, endophthalmitis, retinitis, dysfunctional lens syndrome (DLS), Fuch's Dystrophy, Sjögren's Syndrome, Stargardt disease, cataract, diabetic retinopathy, ocular hypertension, optic neuritis, corneal dystrophies, keratitis, scleritis, amblyopia, strabismus, blepharitis, blepharospasm, central retinal vein occlusion, coloboma, ocular histoplasmosis syndrome, conjunctivitis, presbyopia, or dry eye in a subject in need thereof, the method comprising administering a hyaluronic acid disclosed herein or a composition disclosed herein to one or both eyes of the subject, or implanting a composition disclosed herein into one or both eyes of the subject.

In some aspects, the composition is administered to or implanted in the anterior chamber, posterior chamber, suprachoroidal space, or vitreous humor of one or both eyes of the subject.

In some aspects, the glaucoma is open-angle glaucoma, angle-closure glaucoma, congenital glaucoma, or secondary glaucoma.

### BRIEF DESCRIPTION OF THE DRAWINGS

FIG. 1 is a line graph depicting the maximum pupil diameter over time in beagle dogs intravitreally injected with a bimatoprost acid depot or vehicle.

FIG. 2 is a line graph depicting the intraocular pressure change from baseline over time in beagle dogs intravitreally injected with a bimatoprost acid depot or vehicle.

FIG. 3 is a line graph depicting the intraocular pressure over time in beagle dogs intravitreally injected with a bimatoprost acid depot or vehicle.

FIG. 4 is a line graph depicting the intraocular pressure change from baseline over time in beagle dogs suprachoroidally injected with a bimatoprost acid depot or vehicle.

FIG. 5 is a line graph depicting the maximum pupil diameter over time in beagle dogs suprachoroidally injected with a bimatoprost acid depot or vehicle.

FIG. 6 is a line graph depicting Hackett McDonald tolerability scores over time in beagle dogs suprachoroidally injected with a bimatoprost acid depot or vehicle.

### DETAILED DESCRIPTION OF THE INVENTION

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

To further define this disclosure, the following terms and definitions are provided.

The term “active pharmaceutical ingredient,” as used herein, refers to any substance that is intended for incorporation into a finished drug product and is intended to furnish pharmacological activity or other direct effect in the diagnosis, cure, mitigation, treatment, or prevention of disease, or to affect the structure or any function of the body. Substances include, but are not limited to, small molecules, peptides, and biologics, e.g., antibodies.

The term “finished drug product,” as used herein, refers to a finished dosage form (e.g., tablet, capsule, or solution) that contains at least one active pharmaceutical ingredient, generally, but not necessarily, in association with other ingredients in finished package form suitable for distribution to pharmacies, hospitals, or other sellers or dispensers of the drug product to patients or consumers.

The term “drug delivery system,” as used herein, refers to a composition that is capable of releasing one or more active pharmaceutical ingredients at a known and/or controlled rate.

The terms “pharmaceutical formulation,” “composition,” and “pharmaceutical composition,” as used herein, can be used interchangeably to refer to a preparation which is in such form as to permit the biological activity of one or more active pharmaceutical ingredient(s) to be effective, and which contains no additional components which are unacceptably toxic to a subject to which the formulation would be administered. The pharmaceutical formulation can be sterile.

The term “pharmaceutically acceptable,” as used herein, refers to those compounds, materials, compositions, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Note that something can be pharmaceutically acceptable even if there are very rare instances of hypersensitivity to administration of any compounds, materials, compositions, formulations, and/or dosage forms.

The term “excipient” refers to any substance, not itself an active pharmaceutical ingredient, which may be used in a composition for delivery of an active pharmaceutical ingredient to a subject or combined with an active pharmaceutical ingredient (e.g., to create a pharmaceutical composition) to improve its handling or storage properties or to permit or facilitate formation of a dose unit of the composition. Excipients include, but are not limited to, solvents, penetration enhancers, wetting agents, antioxidants, lubricants, emollients, substances added to improve appearance or texture of the composition and substances used to form hydrogels. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of one or more active pharmaceutical ingredients. The excipient can be an inert substance, an inactive substance, and/or a not medicinally active substance. The excipient can serve various purposes. A person skilled in the art can select one or more excipients with respect to the particular desired properties by routine experimentation and without any undue burden. The amount of each excipient used can vary within ranges conventional in the art. Techniques and excipients which can be used to formulate dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005).

The term “dose unit of the composition,” as used herein, refers to a drug delivery system containing a quantity of one or more active pharmaceutical ingredient(s), said quantity being such that one or more predetermined units may be provided as a single therapeutic administration.

The terms “administer,” “administering,” “administration,” and the like, as used herein, refer to methods that may be used to enable delivery of one or more active pharmaceutical ingredient(s), e.g., bimatoprost acid, to the desired site of biological action (e.g., to the eye). Administration techniques that can be employed with the active pharmaceutical ingredients and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

As used herein, the terms “subject” and “patient” are used interchangeably. The subject can be an animal. In some aspects, the subject is a mammal such as a non-human animal (e.g., cow, pig, horse, cat, dog, rat, mouse, monkey or other primate, etc.). In some aspects, the subject is a human.

The term “therapeutically effective amount” refers to an amount of an active pharmaceutical ingredient effective to treat a disease or disorder in a subject. A “prophylactically effective amount” refers to an amount effective to achieve the desired prophylactic result.

Terms such as “treating,” “treatment,” “to treat,” “alleviating,” and “to alleviate” refer to therapeutic measures that cure, slow down, lessen one or more symptoms of, and/or halt progression of a pathologic condition or disorder. Thus, those in need of treatment include those already diagnosed with or suspected of having the disorder.

As used herein, the term “bound active pharmaceutical ingredient” and “covalently bonded active pharmaceutical ingredient” refers to an active pharmaceutical ingredient that is linked to a drug delivery system, e.g., hyaluronic acid. In some aspects, the active pharmaceutical ingredient is linked to the drug delivery system via a covalent bond.

As used herein, the term “free active pharmaceutical ingredient” refers to an active pharmaceutical ingredient that is not linked to a drug delivery system. For example, an active pharmaceutical ingredient dissolved in a solution without any additional components would be considered a free active pharmaceutical ingredient.

As used herein, the term “hydrophobic group,” as used herein, refers to a chemical fragment that is substantially non-polar and imparts decreased water solubility on a larger compound and/or polymer to which the fragment is attached. Non-limiting examples of hydrophobic groups include alkyl groups, cycloalkyl groups, and phenyl groups.

The term “alkyl,” as used herein, by itself or as part of another group refers to a straight- or branched-chain aliphatic hydrocarbon containing one to twelve carbon atoms, i.e., a C₁-C₁₂ alkyl, or the number of carbon atoms designated, e.g., C₁-C₃ alkyl such as methyl, ethyl, propyl, or isopropyl; a C₁-C₄ alkyl such as methyl, ethyl, propyl, isopropyl, n-butyl, isobutyl, sec-butyl, or t-butyl; and so on. In one embodiment the alkyl is a straight-chain alkyl. In another embodiment, the alkyl is a branched-chain alkyl. In one embodiment, the alkyl is a C₁-C₈ alkyl. In another embodiment, the alkyl is a C₁-C₆ alkyl. In another embodiment, the alkyl is a C₁-C₄ alkyl. In another embodiment, the alkyl is a C₁-C₃ alkyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl.

The term “optionally substituted alkyl,” as used herein, by itself or as part of another group refers to an alkyl group that is either unsubstituted or substituted with one to three substituents, wherein the substituents are each independently halogen, nitro, cyano, hydroxyl, amino, alkoxy, alkylthio, or cycloalkyl.

The term “halo” or “halogen,” as used herein, by itself, or as part of another group, refers to —Cl, —F, —Br, or —I.

The term “nitro,” as used herein, by itself, or as part of another group, refers to —NO₂.

The term “cyano,” as used herein, by itself, or as part of another group, refers to —CN.

The term “hydroxyl,” as used herein, by itself, or as part of another group, refers to —OH.

The term “amino,” as used by itself, or as part of another group, refers to a radical of the formula —NR³R⁴, wherein R³ and R⁴ are independently hydrogen or alkyl. In some aspects, the amino is —NH₂. In some aspects, the amino is an “alkylamino,” i.e., an amino group wherein R³ is C₁₋₆ alkyl and R⁴ is hydrogen. In some aspects, the amino is a “dialkylamino,” i.e., an amino group wherein R³ and R⁴ are independently C₁₋₆ alkyl.

The term “alkoxy,” as used herein, by itself, or as part of another group, refers to an optionally substituted alkyl attached to a terminal oxygen atom. In some aspects, the optionally substituted alkyl is a C₁-C₆ alkyl and the resulting alkoxy is referred to as a “C-₁-C₆ alkoxy.” In some aspects, the optionally substituted alkyl is a C₁-C₄ alkyl group and thus the resulting alkoxy is referred to as a “C₁-C₄ alkoxy.” Non-limiting exemplary alkoxy groups include methoxy, ethoxy, and tert-butoxy.

The term “alkylthio,” as used herein, by itself, or as part of another group, refers to an optionally substituted alkyl group attached to a terminal sulfur atom. In some aspects, the optionally substituted alkyl is a C₁-C₆ alkyl and the resulting alkylthio is referred to as a “C₁-C₆ alkylthio.” In some aspects, the optionally substituted alkyl group is a C₁-C₄ alkyl group and the resulting alkylthio is referred to as a “C₁-C₄ alkylthio.” Non-limiting exemplary alkylthio groups include —SCH₃, and —SCH₂CH₃.

The term “cycloalkyl,” as used herein, by itself, or as part of another group, refers to saturated and partially unsaturated, e.g., containing one or two double bonds, monocyclic, bicyclic, or tricyclic aliphatic hydrocarbons containing three to twelve carbon atoms, i.e., a C₃-C₁₂ cycloalkyl, or the number of carbons designated, e.g., a C₃-C₆ cycloalkyl such a cyclopropyl, cyclobutyl, cyclopentyl, and cyclohexyl. In one embodiment, the cycloalkyl is bicyclic, i.e., it has two rings. In another embodiment, the cycloalkyl is monocyclic, i.e., it has one ring. In another embodiment, the cycloalkyl is a C₃-C₈ cycloalkyl. In another embodiment, the cycloalkyl is a C₃-C₆ cycloalkyl. In another embodiment, the cycloalkyl is a C₅ cycloalkyl, i.e., cyclopentyl. In another embodiment, the cycloalkyl is a C₆ cycloalkyl, i.e., cyclohexyl. Non-limiting exemplary C₃-C₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “optionally substituted cycloalkyl,” as used herein, by itself, or as part of another group, refers to a cycloalkyl group that is either unsubstituted or substituted with one to three substituents, wherein the substituents are each independently halogen, nitro, cyano, hydroxyl, alkyl, amino, alkoxy, or alkylthio.

As used in the present disclosure and claims, the singular forms “a,” “an,” and “the” include plural forms unless the context clearly dictates otherwise.

The term “comprising,” as used herein, is inclusive or open-ended and does not exclude additional, unrecited elements or method steps, and is synonymous with “including,” “containing,” or “characterized by.”

The term “consisting essentially of,” as used herein, limits the scope of a claim to the specified materials or steps and those that do not materially affect the basic and novel characteristic(s) of the claimed invention.

The term “consisting of,” as used herein, excludes any element, step, or ingredient not specified in the claim.

It is understood that wherever aspects are described herein with the language “comprising,” otherwise analogous aspects described in terms of “consisting of” and/or “consisting essentially of” are also provided.

Unless specifically stated or obvious from context, as used herein, the term “or” is understood to be inclusive. The term “and/or” as used in a phrase such as “A and/or B” herein is intended to include any of “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of 10% above and 10% below the value or range remain within the intended meaning of the recited value or range.

### DESCRIPTION

The present disclosure provides physical blends of hyaluronic acid to form a drug delivery system in the form of an injectable depot that can sustain the delivery of small molecules and/or peptides to the eye as well as sequester particulates in the vitreous. Varying degrees of functionalization with hydrophobic groups and/or crosslinking may be utilized to control the depot erosion. In some embodiments, the HA drug delivery system also comprises collagen.

Hyaluronic acid has three functional groups amenable to conjugation: an amide, a carboxylic acid and an alcohol. The carboxylic acid group can be used to synthesize both amide and ester based conjugates. In this disclosure, hyaluronic acid that has not been functionalized or conjugated with hydrophobic group(s) and/or active pharmaceutical ingredients is referred to as “unsubstituted hyaluronic acid.”

The present disclosure provides hyaluronic acid comprising a compound of Formula (I):

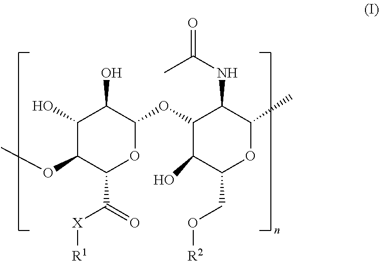

or a pharmaceutically acceptable salt or solvate thereof, wherein:
n is an integer from 500 to 25,000;
X at each occurrence is independently —NH— or —O—;
R¹ and R² at each occurrence are independently selected from the group consisting of hydrogen, a hydrophobic group, -L¹-L²-A, and -A;
each L¹ is an amino acid or absent;
each L² is a linker; and
each A is independently a bound active pharmaceutical ingredient, wherein each instance of A can be the same or different.

In some aspects, n is from 1,200 to 5,000.

In some aspects, X is —NH—. In some aspects, X is —O—.

In some aspects, the hydrophobic group is selected from the group consisting of optionally substituted C₁-C₁₈ alkyl and an amino acid.

In some aspects, the hydrophobic group is C₁-C₁₈ alkyl substituted with a phenyl group. In some aspects, the hydrophobic group is C₁-C₁₈ alkyl that is selected from the group consisting of:

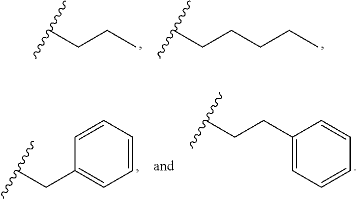

In some aspects, the hydrophobic group is an amino acid that is selected from the group consisting of:

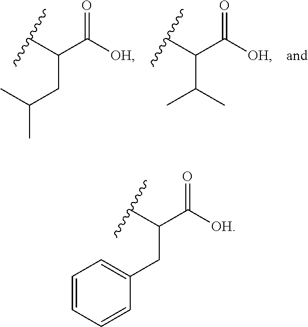

In some aspects, L¹ is selected from the group consisting of:

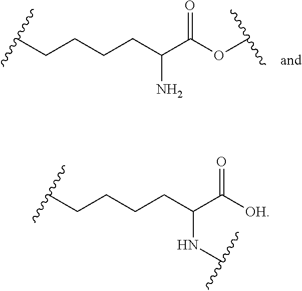 [SMILES: *CCCCC(N)C(=O)O* ; *CCCCC(N*)C(=O)O]

In some aspects, L² is selected from the group consisting of:

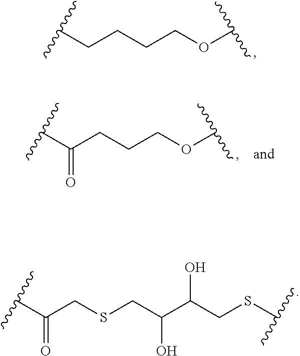 [SMILES: *CCCCO* ; *OCCCC(*)=O ; *SCC(O)C(O)CSCC(*)=O]

In some aspects, each -A of Formula (I) is independently selected from the group consisting of an intraocular pressure (IOP) lowering agent, a ciliary neurotrophic factor (“CNTF”) analog, a FAS inhibitor (also known as a FAS ligand inhibitor), a HIF-1 alpha inhibitor, a HIF-2 alpha inhibitor, a vascular endothelial growth factor (“VEGF”) inhibitor, a vasoactive intestinal peptide (“VIP”) analog, a histatin analog, an alpha crystallin aggregation inhibitor, a reducing agent, a mitochondrial stabilizer, and a tumor necrosis factor-alpha (“TNF-alpha”) inhibitor.

IOP lowering agents include, but are not limited to, prostaglandins, beta blockers, alpha agonists, carbonic anhydrase inhibitors, cholinergic agents, rho kinase inhibitors, and cannabinoid receptor agonists.

Non-limiting examples of prostaglandins include prostaglandin compounds, prostaglandin EP2 agonists, prostaglandin EP3 agonists, and nitric oxide donating prostaglandin compounds. Non-limiting examples of prostaglandin compounds include bimatoprost, bimatoprost acid, travoprost, travoprost acid, latanoprost, latanoprost acid, latanoprostene, tafluprost, and tafluprost acid. Non-limiting examples of prostaglandin EP2 agonists include taprenepag and omidenepag isopropyl. Non-limiting examples of prostaglandin EP3 agonists include dinoprostone, misoprostol, limaprost, gemeprost, alprostadil, and rivenprost. Non-limiting examples of nitric oxide donating prostaglandin compounds include latanoprostene bunod.

Non-limiting examples of beta blockers include timolol, betaxolol, levobunolol, and metipranolol.

Non-limiting examples of alpha agonists include brimonidine and apraclonidine.

Non-limiting examples of carbonic anhydrase inhibitors include brinzolamide, acetazolamide, dorzolamide, and methazolamide.

Non-limiting examples of cholinergic agents include pilocarpine and carbachol.

Non-limiting examples of rho kinase inhibitors include netarsudil and ripasudil.

Non-limiting examples of CNTF analogs include SEQ ID NO: 18, SEQ ID NO.: 1, SEQ ID NO: 2, recombinant CNTF, a compound having a structure or activity similar to ciliary neurotrophic factor (CNTF), or a neurotrophic peptide identified in U.S. Pat. No. 8,592,374, which is incorporated herein by reference.

Non-limiting examples of FAS inhibitors include bicyclol, FLIP, MET12 (SEQ ID NO: 3), compound 1 (SEQ ID NO: 5), compound 2 (SEQ ID NO: 6), compound 3 (SEQ ID NO: 7), compound 4 (SEQ ID NO: 8), compound 5 (SEQ ID NO: 9), compound 6 (SEQ ID NO: 10), compound 7 (SEQ ID NO: 11), compound 8 (SEQ ID NO: 12), compound 9 (SEQ ID NO: 13), compound 10 (SEQ ID NO: 14), compound 11 (SEQ ID NO: 15), ONL1204, SEQ ID NO: 4, FAIM, NOL3, human decoy receptor 1 (DcRl), human decoy receptor 2 (DcR2), and human decoy receptor 3 (DcR3).

Non-limiting examples of TNF-alpha inhibitors include etanercept, infliximab, golimumab, certolizumab, adalimumab, TNFRl-selective antagonistic mutant TNF (RlantTNF), DMS5540, TNF Receptor-One Silencer (TROS), and ATROSAB.

Non-limiting examples of mitochondrial stabilizers include elamipretide and risuteganib.

Non-limiting examples of cannabinoid receptor agonists include Δ⁹-tetrahydrocannabinol (Δ⁹-THC), anandamide, 2-arachidonoyl glycerol, 1,1-dimethylheptyl-11-hydroxy-tetrahydrocannabinol (HU-210), (−)-cis-3-[2-hydroxy-4-(1,1-dimethylheptyl)phenyl]-trans-4-(3-hydroxypropyl)cyclohexanol (CP55940), (R)-(+)-[2,3-dihydro-5-methyl-3-(4-morpholinylmethyl)pyrrolo-[1,2,3-de]-1,4-benzoxazin-6-yl]-1-naphthalenylmethanone (R—(+)-WIN55212), (2-methyl-1-propyl-1H-indol-3-yl)-1-naphthalenylmethanone (JWH-015), 3-(1,1-dimethylbutyl)-6,6,9-trimethyl-6a,7,10,10a-tetrahydro-6H-benzo[c]chromene (JWH-133), onternabez (HU-308), 1-(methylpiperidin-2-ylmethyl)-3-(2-iodo-5-nitrobenzoyl)indole (AM1241), 1-(2,3-Dichlorobenzoyl)-5-methoxy-2-methyl-3-[2-(4-morpholinyl)ethyl]-1H-indole (GW405833), 2-[(2,4-dichlorophenyl)amino]-N-[(tetrahydro-2H-pyran-4-yl)methyl]-4-(trifluoromethyl)-5-pyrimidinecarboxamide (GW842166X), and 1-(4-(1,1-dimethylheptyl)-2,6-dimethoxyphenyl)-3-methylcyclohexanol (O-1966).

In some aspects, each -A of the Formula (I) is independently bimatoprost, bimatoprost acid, brimonidine, netarsudil, latanoprost, tafluprost, travoprost, latanoprostene bunod, dorzolamide, brinzolamide, echothiophate iodide, unoprostone, dipivefrin, pilocarpine, metipranolol, omidenepag isopropyl, carteolol, carbachol, acetylcholine, apraclonidine, betaxolol, timolol, levobunolol, difluprednate, loteprednol, methylprednisolone acetate, triamcinolone acetate, betamethasone, triamcinolone hexacetonide, dexamethasone, fluocinolone, fluorometholone, triamcinolone, rimexolone, prednisolone, lifitegrast, cyclosporine, bromfenac, nepafenac, ketorolac, diclofenac, suprofen, flurbiprofen, moxifloxacin, gatifloxacin, ganciclovir, azithromycin, besifloxacin, tobramycin, trifluridine, vidarabine, erythromycin, levofloxacin, ofloxacin, gentamicin, chloramphenicol, natamycin, bacitracin, tropicamide, phenylephrine, homatropine, cyclopentolate, or atropine.

In some aspects, each -A of Formula (I) is independently a compound or peptide disclosed in WO 2019/246130 A1, WO 2021/127124 A1, WO 2019/246141 A1, and/or WO 2021/127052 A1, the contents of which are herein incorporated by reference.

In some aspects, from about 1% to about 50% of the R¹ and R² groups of Formula (I) are hydrophobic groups. In some aspects, from about 1% to about 50% of the R¹ and R² groups of Formula (I) are -L¹-L²-A or -A. In any of these aspects, each instance of the group “-A” in Formula (I) may be the same or different. In some aspects, there is only one active pharmaceutical ingredient denoted by -A in Formula (I). In some aspects, there are two active pharmaceutical ingredients denoted by -A in Formula (I). In some aspects, there are three active pharmaceutical ingredients denoted by -A in Formula (I). When there is more than one active pharmaceutical ingredient denoted by -A in Formula (I), the ratio of active pharmaceutical ingredients may be in any stoichiometric ratio appropriate for treatment of disease.

In some aspects, there are two or more different active pharmaceutical ingredients denoted by -A in Formula (I). In some aspects, multiple different active pharmaceutical ingredients (denoted by -A) can be added to the hyaluronic acid by sequentially reacting the hyaluronic acid with an appropriate quantity of each active pharmaceutical ingredient. For example, hyaluronic acid can be reacted with 0.5 molar equivalents of a first active pharmaceutical ingredient to make a first hyaluronic acid-pharmaceutical active ingredient complex. The first hyaluronic acid-pharmaceutical active ingredient complex can then be reacted with 0.5 molar equivalents of a second active pharmaceutical ingredient resulting in a compound of Formula (I) wherein about 50% of the -A groups are the first active pharmaceutical ingredient and about 50% of the -A groups are the second active pharmaceutical ingredient.

In some aspects, a first compound of Formula (I) and one or more additional compounds of Formula (I) are cross-linked with each other to form a cross-linked hyaluronic acid. In some aspects, the first compound of Formula (I) and the one or more additional compounds of Formula (I) are cross-linked via covalent bond(s), e.g., a bond formed from a free hydroxyl of a compound of Formula (I) and a free acid of an additional compound of Formula (I). In some aspects, the first compound of Formula (I) and the one or more additional compounds of Formula (I) are cross-linked via non-covalent interactions, including, but not limited to, hydrophobic interactions, hydrogen bonding, charge interaction, or stereocomplexation. In some aspects, the first compound of Formula (I) and the one or more additional compounds of Formula (I) comprise different active pharmaceutical ingredient(s).

### Amide-Based Conjugates

In some aspects, the compound of Formula (I) is covalently bonded to one or more active pharmaceutical ingredients and/or a hydrophobic group via an amide linkage. The amide linkage may be formed by various methods. In some aspects, a hyaluronate salt, e.g., sodium hyaluronate, is a converted to a more hydrophobic salt, e.g., a tert-butyl ammonium (TBA) salt, which is further reacted with the active pharmaceutical ingredient or hydrophobic group and appropriate peptide coupling agent(s) to form the amide linkage.

In some aspects, sodium hyaluronate is first converted to the tert-butyl ammonium (TBA) salt. Sodium HA with a molecular weight (M.W.) of 500K Da to 2M Da can be converted to the TBA salt by passing through an ion exchange resin, e.g., Amberlite IR-120, in TBA form. The eluate can then be vacuum dried or lyophilized for future use. To synthesize the amide based hydrophobic conjugates, HA TBA is dissolved in a solvent, e.g., an organic solvent, e.g., DMSO. Catalytic amounts of methane sulfonic acid and 1,1′-carbonyldiimidazole can then be added and activated by stirring at room temperature. After 60 minutes, an alkylamine or amino acid can be added and stirred overnight at 42° C. to yield the final product. The amount of alkylamine or amino acid may vary from a 1:2 ratio of alkylamine or amino acid mEq to HA disaccharide unit mEq to a 1:1 ratio and finally a 2:1 ratio. The various ratios will yield different degrees of substitution (see Scheme 1).

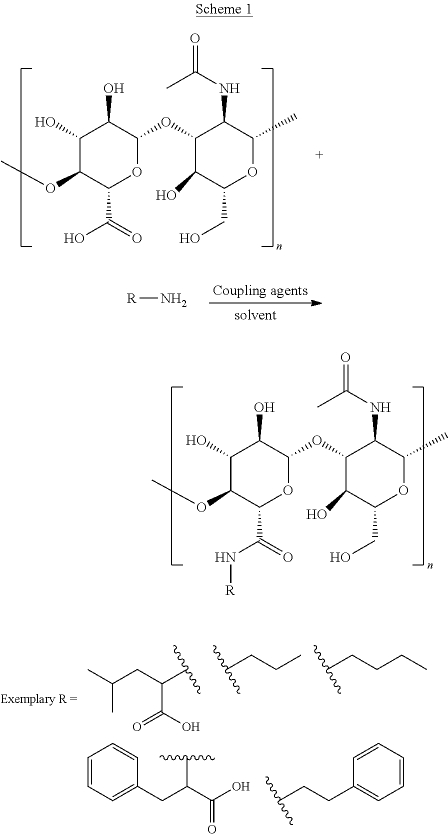

Compounds of Formula (I) comprising hydrophobic amino acid(s) can be synthesized using leucine, phenylalanine and/or other hydrophobic, non-natural amino acids. HA TBA can be dissolved in a solvent, e.g., an organic solvent, e.g., DMSO, to which a carboxyl activating agent, EDC (1-Ethyl-3-[3-dimethylaminopropyl]carbodiimide hydrochloride) is added, and NHS (N-hydroxysuccinimide) is added to form an amine reactive NHS ester of HA. The reactive NHS HA ester can then be added to a solution of a hydrophobic amino acid to prepare the compound of Formula (I).

### Ester-Based Conjugates

In some aspects, the compound of Formula (I) can be covalently bound to an active pharmaceutical ingredient and/or hydrophobic group via an ester linkage. The ester linkage may be formed by various methods. In some aspects, a hyaluronate salt, e.g., sodium hyaluronate, is converted to a more hydrophobic salt, e.g., a tert-butyl ammonium (TBA) salt, which is further reacted with the active pharmaceutical ingredient or hydrophobic group, e.g., an active pharmaceutical ingredient or hydrophobic group comprising an alkyl halide, to form the ester linkage.

Sodium hyaluronate can first be converted to the tert-butyl ammonium (TBA) salt to increase the solubility in the solvents, e.g., organic solvents, used in the synthesis of the conjugates. To synthesize the ester based hydrophobic conjugates, HA TBA can be dissolved in a suitable amount of a solvent, e.g., an organic solvent, e.g., DMSO. The appropriate amount of alkyl halide can be added with stirring at 30° C. for 12 hours to form the ester. The amount of alkyl halide used in the reaction will vary from a 1:2 ratio of alkyl halide mEq to HA disaccharide unit mEq to a 1:1 ratio and finally a 2:1 ratio. The various ratios will yield different degrees of substitution (see Scheme 2).

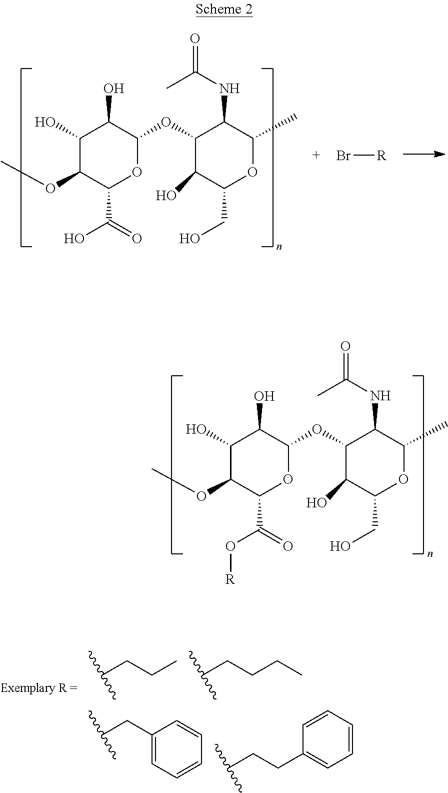

Hydrophobic substituents can be derived from the homologous series of methyl halide (e.g. methyl bromide) to octadecyl halide and benzyl, ethylphenyl and propylphenyl halides.

### In Situ Gelling Hydrophobic HA Depots

The hydrophobic HA, e.g., comprising a compound of Formula (I), synthesized in this disclosure can be dissolved in an aqueous solution of active pharmaceutical ingredient at 0.1 to 5% w/w. The concentration of active pharmaceutical ingredient in the aqueous solution can range from 1 to 20% w/w. The final formulation will be a shear thinning hydrogel that can be injected through small bore needles (22 Gauge or smaller). Upon injection into the eye or other biologic system, the hydrophobic HA will self-assemble of micellize, depending on the substituent, and entrap the one or more active pharmaceutical ingredients in solution.

The one or more active pharmaceutical ingredient entrapped in the depot will release into the vitreous over a period of 1 week to 12 months or longer. It is expected that the hydrophobic HA depot should degrade concurrent with active pharmaceutical ingredient release over a period of 1 week to 12 months or longer.

In Situ Depots from Physical Blends of Polymers with Hydrophobic HA

In some aspects, collagen and poloxamers such as Pluronics can be used to enhance the gel formation of HA. In some aspects, by adding these to hydrophobic HA, in situ forming depots which can sustain the release of one or more active pharmaceutical ingredient can be obtained. Collagen or a poloxamer can be added to hydrophobic HA according to table 1 below. A solution of one or more active pharmaceutical ingredient in either collagen or poloxamer can be made. Active pharmaceutical ingredient concentrations can range from about 0.1% to about 50% w/w in the collagen or poloxamer solution. An aqueous hydrogel of hydrophobic HA can be made at an HA concentration of about 0.2 to about 10% w/w. Equal volumes of the active pharmaceutical ingredient solution and the HA solution can then be mixed to form the final formulation.

TABLE 1

| | | |
|---|---|---|
| Hydrophobic HA | Collagen | Pluronic F127 |

| | | | | |
|---|---|---|---|---|
| 1 | mg/mL | 1 | mg/mL | 0 |
| 1 | mg/mL | 2.5 | mg/mL | 0 |
| 1 | mg/mL | 5 | mg/mL | 0 |
| 5 | mg/mL | 1 | mg/mL | 0 |
| 5 | mg/mL | 2.5 | mg/mL | 0 |
| 5 | mg/mL | 5 | mg/mL | 0 |
| 10 | mg/mL | 1 | mg/mL | 0 |
| 10 | mg/mL | 2.5 | mg/mL | 0 |
| 10 | mg/mL | 5 | mg/mL | 0 |

| | | | |
|---|---|---|---|
| 1 | mg/mL | 0 | 150 mg/mL |
| 1 | mg/mL | 0 | 200 mg/mL |
| 1 | mg/mL | 0 | 250 mg/mL |
| 5 | mg/mL | 0 | 150 mg/mL |
| 5 | mg/mL | 0 | 200 mg/mL |
| 5 | mg/mL | 0 | 250 mg/mL |
| 10 | mg/mL | 0 | 150 mg/mL |
| 10 | mg/mL | 0 | 200 mg/mL |
| 10 | mg/mL | 0 | 250 mg/mL |

In some aspects, the concentration of the collagen or poloxamer is from about 0.1 mg/mL to about 10 mg/mL, from about 0.1 mg/mL to about 50 mg/mL, from about 0.1 mg/mL to about 100 mg/mL, from about 0.1 mg/mL to about 200 mg/mL, from about 0.1 mg/mL to about 300 mg/mL, from about 0.1 mg/mL to about 400 mg/mL, from about 0.1 mg/mL to about 500 mg/mL, from about 10 mg/mL to about 50 mg/mL, from about 10 mg/mL to about 100 mg/mL, from about 10 mg/mL to about 200 mg/mL, from about 10 mg/mL to about 300 mg/mL, from about 10 mg/mL to about 400 mg/mL, from about 10 mg/mL to about 500 mg/mL, from about 100 mg/mL to about 200 mg/mL, from about 100 mg/mL to about 300 mg/mL, from about 100 mg/mL to about 400 mg/mL, from about 100 mg/mL to about 500 mg/mL, from about 200 mg/mL to about 300 mg/mL, from about 200 mg/mL to about 400 mg/mL, from about 200 mg/mL to about 500 mg/mL, from about 300 mg/mL to about 400 mg/mL, from about 300 mg/mL to about 500 mg/mL, or from about 400 mg/mL to about 500 mg/mL.

In some aspects, the concentration of the collagen or poloxamer is from 0.1 mg/mL, about 0.5 mg/mL, about 1 mg/mL, about 2 mg/mL, about 3 mg/mL, about 4 mg/mL, about 5 mg/mL, about 10 mg/mL, about 15 mg/mL, about 20 mg/mL, about 25 mg/mL, about 50 mg/mL, about 100 mg/mL, about 150 mg/mL, about 200 mg/mL, about 250 mg/mL, about 300 mg/mL, about 350 mg/mL, about 400 mg/mL, about 450 mg/mL, or about 500 mg/mL.

HA with Cleavable Linkers

In some aspects, the compound of Formula (I) is covalently linked to one or more active pharmaceutical ingredients via a cleavable linker. Release of the one or more active pharmaceutical ingredient(s) can be further tuned and active pharmaceutical ingredient loading increased by linking the desired active pharmaceutical ingredients to the HA backbone. Since it is unknown what affects the HA will have on the disposition or potency of the attached active pharmaceutical ingredients, in some aspects, cleavable linkers may be desired. Utilizing cleavable linkers to covalently bond a hydrophobic or natural HA backbone may allow for further optimization of active pharmaceutical ingredient(s) release from the depot. In one aspect, active pharmaceutical ingredient release becomes a function of the diffusional properties of the depot as well as the kinetics of linker cleavage. Several linkers have been contemplated that can cleave in the vitreous thereby liberating the linked one or more active pharmaceutical ingredients.

Various cleavable linkers can be used to link small molecules, peptides, and/or proteins to an HA backbone. Hydrophobic HA with a degree of substitution of hydrophobic groups on the HA back bone ranging from 0% to 50% molar equivalent of hydrophobic substituent to disaccharide units can be used. Cleavable linkers that can be used include, without limitation, ester linkers, disulfide linkers or a mixture thereof.

### Cleavable HA Linkers Based on Lysine Linkers

In some aspects, the compound of Formula (I) is covalently linked to one or more active pharmaceutical ingredients and/or hydrophobic groups via lysine linkers. The lysine linker may be attached to hyaluronic acid by various methods, e.g., a peptide coupling.

In some aspects, the compound of Formula (I) is conjugated to one or more active pharmaceutical ingredient and/or hydrophobic groups via different types of linkers. Non-limiting examples of linkers include esters, amides, and disulfides. The linkers may comprise an optionally substituted alkyl group or an optionally substituted cycloalkyl group. In some aspects, the linkers are derived from an amino acid, e.g., lysine.

In some aspects, the compound of Formula (I) is prepared by the following process. Sodium hyaluronate is first converted to the tert-butyl ammonium salt (HA TBA). HA can be conjugated with lysine using, e.g., EDC/NHS activation of the HA carboxylic acid. HA TBA is dissolved in a solvent, e.g., DMSO to which a carboxyl activating agent, e.g., EDC, is added, and to which NHS is added to form an amine reactive NHS ester of HA. The reactive NHS HA ester can then be added to a solution of lysine to create the lysine amide of HA. The HA-lysine amide can be reacted with an diol, e.g., 1,4-butanediol, which can then be used to esterify the C-terminus of peptides or carboxylic acid residues of small molecule active pharmaceutical ingredients, e.g., bimatoprost. The HA-lysine amide can also be reacted with a compound comprising an alkyl halide and a carboxylate, e.g., bromobutyrate, which can then be used to esterify the C-terminus of peptides or hydroxyl residues of small molecule active pharmaceutical ingredients. A compound of Formula (I) preparing using 1,4-butanediol is shown in Formula I, while with a compound of Formula (I) prepared using bromobutyric acid is shown in Formula II.

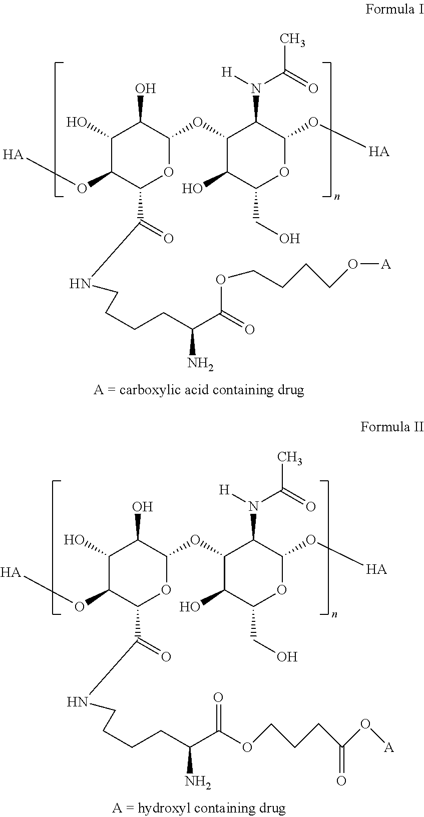

### Direct Ester Based Conjugates

In some aspects, the compound of Formula (I) is prepared by the following process. Sodium hyaluronate is first converted to the tert-butyl ammonium salt (HA TBA). 1 gram of HA TBA is dissolved in an organic solvent, e.g., DMSO. The appropriate amount of alkyl halide, e.g., an alkyl halide linked to an active pharmaceutical ingredient, is added to form an ester. The amount of alkyl halide may vary from about a 1:2 ratio of alkyl halide mEq to HA disaccharide unit mEq to about a 1:1 ratio and finally about a 2:1 ratio. In some aspects, the ratio of alkyl halide mEq to HA disaccharide unit mEq is about 1:3, about 2:5, about 1:2, about 4:7, about 2:3, about 4:5, about 1:1, about 5:4, about 3:2, about 7:4, about 2:1, about 5:2, or about 3:1. The various ratios will yield different degrees of substitution. A compound of Formula (I) linked with alkyl halides is shown in Scheme 3.

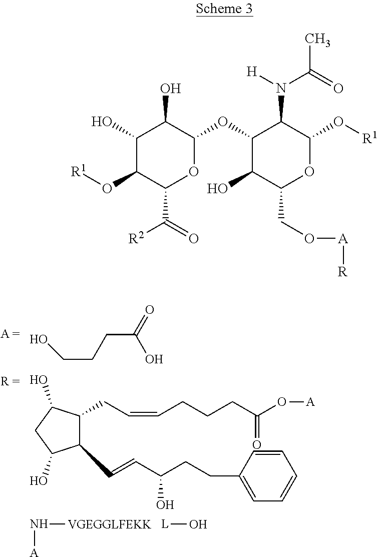

### Disulfide Based Cleavable Linkers

In some aspects, the compound of Formula (I) comprises a cleavable disulfide linker, i.e., L² comprises a disulfide, which links the active pharmaceutical ingredient(s), i.e., -A, to the compound. The disulfide linkage can undergo oxidative cleavage taking advantage of the high reduced glutathione concentration in the eye. This creates a prodrug uniquely suited to the intraocular environment.

In some aspects, a compound of Formula (I) can be synthesized as shown in Scheme 4 below. Briefly, HA-TBA salt is dissolved in an organic solvent (e.g., N,N-dimethylformamide (DMF)), optionally comprising a salt, (e.g., LiCl), and heated until dissolved, then cooled to room temperature. A base (e.g., pyridine) can be added with a halogenated acyl halide (e.g., chloroacetyl chloride) and reacted to form a haloacetyl modified HA. The haloacetyl HA can then be reacted with excess dithiol (e.g., dithiothreitol) in solution (e.g., an aqueous buffered solution). The thiolated HA thus formed can be used to form disulfide prodrugs of sulfhydryl containing compounds, such as cysteine containing peptides. Substituents can be added to the dithiothreitol to control the oxidative cleavage rate.

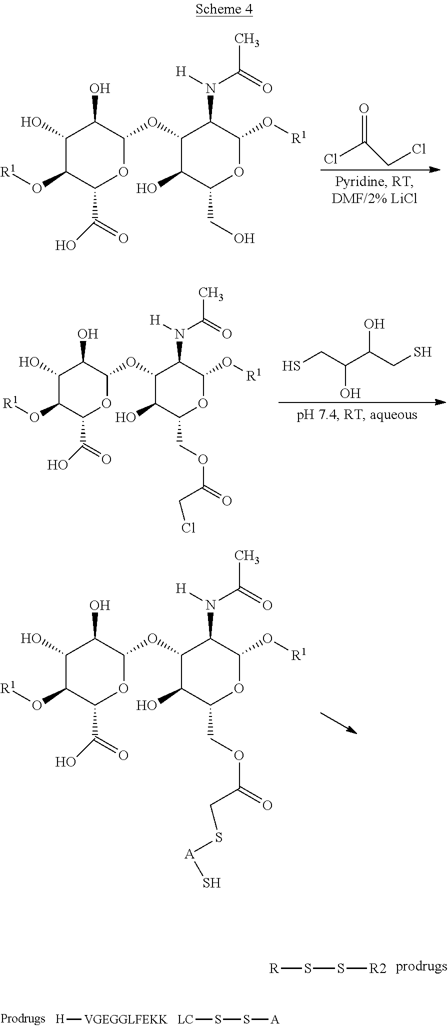

### Compositions

The present disclosure also provides compositions comprising the compound of Formula (I) disclosed herein and one or more free active pharmaceutical ingredient(s).

In some aspects, each of the one or more free active pharmaceutical ingredient(s) is independently selected from the group consisting of an intraocular pressure (IOP) lowering agent, a ciliary neurotrophic factor (“CNTF”) analog, a FAS inhibitor (also known as a FAS ligand inhibitor), a HIF-1 alpha inhibitor, a HIF-2 alpha inhibitor, a vascular endothelial growth factor (“VEGF”) inhibitor, a vasoactive intestinal peptide (“VIP”) analog, a histatin analog, an alpha crystallin aggregation inhibitor, a reducing agent, a mitochondrial stabilizer, and a tumor necrosis factor-alpha (“TNF-alpha”) inhibitor. Non-limiting examples of these agents are provided above.

In some aspects, the one or more free active pharmaceutical ingredient(s) is selected from the group consisting of bimatoprost, bimatoprost acid, brimonidine, netarsudil, latanoprost, tafluprost, travoprost, latanoprostene bunod, dorzolamide, brinzolamide, echothiophate iodide, unoprostone, dipivefrin, pilocarpine, metipranolol, omidenepag isopropyl, carteolol, carbachol, acetylcholine, apraclonidine, betaxolol, timolol, levobunolol, difluprednate, loteprednol, methylprednisolone acetate, triamcinolone acetate, betamethasone, triamcinolone hexacetonide, dexamethasone, fluocinolone, fluorometholone, triamcinolone, rimexolone, prednisolone, lifitegrast, cyclosporine, bromfenac, nepafenac, ketorolac, diclofenac, suprofen, flurbiprofen, moxifloxacin, gatifloxacin, ganciclovir, azithromycin, besifloxacin, tobramycin, trifluridine, vidarabine, erythromycin, levofloxacin, ofloxacin, gentamicin, chloramphenicol, natamycin, bacitracin, tropicamide, phenylephrine, homatropine, cyclopentolate, and atropine.

In some aspects, the individual concentrations of the one or more free active pharmaceutical ingredient(s) is from about 1 wt % to about 20 wt %. In some aspects, the individual concentrations of the one or more free active pharmaceutical ingredient(s) is from about 1 wt % to about 2 wt %, from about 1 wt % to about 4 wt %, from about 1 wt % to about 6 wt %, from about 1 wt % to about 8 wt %, from about 1 wt % to about 10 wt %, from about 1 wt % to about 12 wt %, from about 1 wt % to about 14 wt %, from about 1 wt % to about 16 wt %, from about 1 wt % to about 18 wt %, from about 2 wt % to about 4 wt %, from about 2 wt % to about 6 wt %, from about 2 wt % to about 8 wt %, from about 2 wt % to about 10 wt %, from about 2 wt % to about 12 wt %, from about 2 wt % to about 14 wt %, from about 2 wt % to about 16 wt %, from about 2 wt % to about 18 wt %, from about 2 wt % to about 20 wt %, from about 4 wt % to about 6 wt %, from about 4 wt % to about 8 wt %, from about 4 wt % to about 10 wt %, from about 4 wt % to about 12 wt %, from about 4 wt % to about 14 wt %, from about 4 wt % to about 16 wt %, from about 4 wt % to about 18 wt %, from about 4 wt % to about 20 wt %, from about 6 wt % to about 8 wt %, from about 6 wt % to about 10 wt %, from about 6 wt % to about 12 wt %, from about 6 wt % to about 14 wt %, from about 6 wt % to about 16 wt %, from about 6 wt % to about 18 wt %, from about 6 wt % to about 20 wt %, from about 8 wt % to about 10 wt %, from about 8 wt % to about 12 wt %, from about 8 wt % to about 14 wt %, from about 8 wt % to about 16 wt %, from about 8 wt % to about 18 wt %, from about 8 wt % to about 20 wt %, from about 10 wt % to about 12 wt %, from about 10 wt % to about 14 wt %, from about 10 wt % to about 16 wt %, from about 10 wt % to about 18 wt %, from about 10 wt % to about 20 wt %, from about 12 wt % to about 14 wt %, from about 12 wt % to about 16 wt %, from about 12 wt % to about 18 wt %, from about 12 wt % to about 20 wt %, from about 14 wt % to about 16 wt %, from about 14 wt % to about 18 wt %, from about 14 wt % to about 20 wt %, from about 16 wt % to about 18 wt %, from about 16 wt % to about 20 wt %, or from about 18 wt % to about 20 wt %. In any of the previous aspects, the total concentration of all the individual free active pharmaceutical ingredients is from about 1 wt % to about 50 wt %.

In some aspects, the concentration of each individual free active pharmaceutical ingredient is about 5 wt %. In some aspects, the concentration of each individual free active pharmaceutical ingredient is about 1 wt %, about 2 wt %, about 3 wt %, about 4 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt %.

In some aspects, the concentration of the hydrophobic groups on the hyaluronic acid is from about 0.1 wt % to about 50 wt % relative to the total weight of the hyaluronic acid without the active pharmaceutical ingredient (-A) in Formula (I). In some aspects, the concentration of the hydrophobic groups on the hyaluronic acid is from about 0.1 wt % to about 0.5 wt %, from about 0.1 wt % to about 1 wt %, from about 0.1 wt % to about 2 wt %, from about 0.1 wt % to about 3 wt %, from about 0.1 wt % to about 4 wt %, from about 0.5 wt % to about 1 wt %, from about 0.5 wt % to about 2 wt %, from about 0.5 wt % to about 3 wt %, from about 0.5 wt % to about 4 wt %, from about 0.5 wt % to about 5 wt %, from about 1 wt % to about 2 wt %, from about 1 wt % to about 3 wt %, from about 1 wt % to about 4 wt %, from about 1 wt % to about 5 wt %, from about 2 wt % to about 3 wt %, from about 2 wt % to about 4 wt %, from about 2 wt % to about 5 wt %, from about 3 wt % to about 4 wt %, from about 3 wt % to about 5 wt %, or from about 4 wt % to about 5 wt % relative to the total weight of the hyaluronic acid without the active pharmaceutical ingredient (-A) in Formula (I). In some aspects, the concentration of the hydrophobic groups on the hyaluronic acid is from about 1 wt % to about 10 wt %, from about 1 wt % to about 20 wt %, from about 1 wt % to about 30 wt %, from about 1 wt % to about 40 wt %, from about 1 wt % to about 50 wt %, from about 10 wt % to about 20 wt %, from about 10 wt % to about 30 wt %, from about 10 wt % to about 40 wt %, from about 10 wt % to about 50 wt %, from about 20 wt % to about 30 wt %, from about 20 wt % to about 40 wt %, from about 20 wt % to about 50 wt %, from about 30 wt % to about 40 wt %, from about 30 wt % to about 50 wt %, or from about 40 wt % to about 50 wt % relative to the total weight of the hyaluronic acid without the active pharmaceutical ingredient (-A) in Formula (I).

In some aspects, the concentration of the hydrophobic groups on the hyaluronic acid is about 2 wt % relative to the total weight of the hyaluronic acid without the active pharmaceutical ingredient (-A) in Formula (I). In some aspects, the concentration of the hydrophobic groups on the hyaluronic acid is about 0.5 wt %, about 1 wt %, about 1.5 wt %, about 2.5 wt %, about 3 wt %, about 3.5 wt %, about 4 wt %, about 4.5 wt %, or about 5 wt % relative to the total weight of the hyaluronic acid without the active pharmaceutical ingredient (-A) in Formula (I). In some aspects, the concentration of the hydrophobic groups on the hyaluronic acid is about 10 wt %, about 15 wt %, about 20 wt %, about 25 wt %, about 30 wt %, about 35 wt %, about 40 wt %, about 45 wt %, or about 50 wt % relative to the total weight of the hyaluronic acid without the active pharmaceutical ingredient (-A) in Formula (I).

In some aspects, the composition further comprises a solvent, e.g., water. In some aspects, the hyaluronic acid is dissolved in the solvent.

In some aspects, the concentration of the hyaluronic acid comprising a compound of Formula (I) in the composition is from about 1 mg/mL to about 10 mg/mL. In some aspects, the concentration of the hyaluronic acid in the composition is from about 1 mg/mL to about 2.5 mg/mL, from about 1 mg/mL to about 5 mg/mL, from about 1 mg/mL to about 7.5 mg/mL, from about 2.5 mg/mL to about 5 mg/mL, from about 2.5 mg/mL to about 7.5 mg/mL, from about 2.5 mg/mL to about 10 mg/mL, from about 5 mg/mL to about 7.5 mg/mL, from about 5 mg/mL to about 10 mg/mL, or from about 7.5 mg/mL to about 10 mg/mL.

In some aspects, the concentration of the hyaluronic acid comprising a compound of Formula (I) in the composition is about 5 mg/mL. In some aspects, the concentration of the hyaluronic acid in the composition is about 1 mg/mL, about 2 mg/mL, about 3 mg/mL, about 4 mg/mL, about 6 mg/mL, about 7 mg/mL, about 8 mg/mL, about 9 mg/mL, or about 10 mg/mL.

In some aspects, the hyaluronic acid forms a hydrogel in the composition.

In some aspects, the composition comprises hyaluronic acid in the form of a solid implant.

In some aspects, the composition further comprises a second polymer. In some aspects, the second polymer is a collagen or a poloxamer. In some aspects, the poloxamer is Pluronic® L64, Pluronic® P65, Pluronic® P84, Pluronic® P85, Pluronic® F88, Pluronic® P103, Pluronic® P104, Pluronic® P105, Pluronic® F108, Pluronic® F123, or Pluronic® F127. In some aspects, the collagen is VitriCol®.

In some aspects, the concentration of the second polymer is from about 1 mg/mL to about 250 mg/mL. In some aspects, the concentration of the second polymer is from about 1 mg/mL to about 5 mg/mL. In some aspects, the concentration of the second polymer is from about 1 mg/mL to about 10 mg/mL, from about 1 mg/mL to about 50 mg/mL, from about 1 mg/mL to about 100 mg/mL, from about 1 mg/mL to about 150 mg/mL, from about 1 mg/mL to about 200 mg/mL, from about 5 mg/mL to about 10 mg/mL, from about 5 mg/mL to about 50 mg/mL, from about 5 mg/mL to about 100 mg/mL, from about 5 mg/mL to about 150 mg/mL, from about 5 mg/mL to about 200 mg/mL, from about 5 mg/mL to about 250 mg/mL, from about 10 mg/mL to about 50 mg/mL, from about 10 mg/mL to about 100 mg/mL, from about 10 mg/mL to about 150 mg/mL, from about 10 mg/mL to about 200 mg/mL, from about 10 mg/mL to about 250 mg/mL, from about 50 mg/mL to about 100 mg/mL, from about 50 mg/mL to about 150 mg/mL, from about 50 mg/mL to about 200 mg/mL, from about 50 mg/mL to about 250 mg/mL, from about 100 mg/mL to about 150 mg/mL, from about 100 mg/mL to about 200 mg/mL, from about 100 mg/mL to about 250 mg/mL, from about 150 mg/mL to about 200 mg/mL, from about 150 mg/mL to about 250 mg/mL, or from about 200 mg/mL to about 250 mg/mL.

In some aspects, the concentration of the second polymer is about 5 mg/mL. In some aspects, the concentration of the second polymer is about 1 mg/mL, about 10 mg/mL, about 25 mg/mL, about 50 mg/mL, about 75 mg/mL, about 100 mg/mL, about 125 mg/mL, about 150 mg/mL, about 175 mg/mL, about 200 mg/mL, about 225 mg/mL, or about 250 mg/mL.

In some aspects, the concentration of the second polymer is from about 0.1 wt % to about 25 wt %. In some aspects, the concentration of the second polymer is from about 0.1 wt % to about 0.5 wt %. In some aspects, the concentration of the second polymer is from about 0.1 wt % to about 1 wt %, from about 0.1 wt % to about 5 wt %, from about 0.1 wt % to about 10 wt %, from about 0.1 wt % to about 15 wt %, from about 0.1 wt % to about 20 wt %, from about 0.5 wt % to about 1 wt %, from about 0.5 wt % to about 5 wt %, from about 0.5 wt % to about 10 wt %, from about 0.5 wt % to about 15 wt %, from about 0.5 wt % to about 20 wt %, from about 0.5 wt % to about 25 wt %, from about 1 wt % to about 5 wt %, from about 1 wt % to about 10 wt %, from about 1 wt % to about 15 wt %, from about 1 wt % to about 20 wt %, from about 1 wt % to about 25 wt %, from about 5 wt % to about 10 wt %, from about 5 wt % to about 15 wt %, from about 5 wt % to about 20 wt %, from about 5 wt % to about 25 wt %, from about 10 wt % to about 15 wt %, from about 10 wt % to about 20 wt %, from about 10 wt % to about 25 wt %, from about 15 wt % to about 20 wt %, from about 15 wt % to about 25 wt %, or from about 20 wt % to about 25 wt %.

In some aspects, the concentration of the second polymer is about 0.5 wt %. In some aspects, the concentration of the second polymer is about 0.1 wt %, about 1 wt %, about 2.5 wt %, about 5 wt %, about 7.5 wt %, about 10 wt %, about 12.5 wt %, about 15 wt %, about 17.5 wt %, about 20 wt %, about 22.5 wt %, or about 25 wt %.

In some aspects, the one or more covalently bonded active pharmaceutical ingredient(s) is released over a period of from about 6 months to about 24 months. In some aspects, the one or more covalently bonded active pharmaceutical ingredient(s) is released over a period of from about 6 months to about 8 months, from about 6 months to about 10 months, from about 6 months to about 12 months, from about 6 months to about 14 months, from about 6 months to about 16 months, from about 6 months to about 18 months, from about 6 months to about 20 months, from about 6 months to about 22 months, from about 8 months to about 10 months, from about 8 months to about 12 months, from about 8 months to about 14 months, from about 8 months to about 16 months, from about 8 months to about 18 months, from about 8 months to about 20 months, from about 8 months to about 22 months, from about 8 months to about 24 months, from about 10 months to about 12 months, from about 10 months to about 14 months, from about 10 months to about 16 months, from about 10 months to about 18 months, from about 10 months to about 20 months, from about 10 months to about 22 months, from about 10 months to about 24 months, from about 12 months to about 14 months, from about 12 months to about 16 months, from about 12 months to about 18 months, from about 12 months to about 20 months, from about 12 months to about 22 months, from about 12 months to about 24 months, from about 14 months to about 16 months, from about 14 months to about 18 months, from about 14 months to about 20 months, from about 14 months to about 22 months, from about 14 months to about 24 months, from about 16 months to about 18 months, from about 16 months to about 20 months, from about 16 months to about 22 months, from about 16 months to about 24 months, from about 18 months to about 20 months, from about 18 months to about 22 months, from about 18 months to about 24 months, from about 20 months to about 22 months, from about 20 months to about 24 months, or from about 22 months to about 24 months.

In some aspects, the one or more covalently bonded active pharmaceutical ingredient(s) is released over a period of about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months. In some aspects, the one or more covalently bonded active pharmaceutical ingredient(s) is released over a period of about 6 months. In some aspects, the one or more covalently bonded active pharmaceutical ingredient(s) is released over a period of about 12 months. In some aspects, the one or more covalently bonded active pharmaceutical ingredient(s) is released over a period of about 18 months. In some aspects, the one or more covalently bonded active pharmaceutical ingredient(s) is released over a period of about 24 months.

In some aspects, the one or more free active pharmaceutical ingredient(s) is released over a period of from about 6 months to about 24 months. In some aspects, the one or more covalently bonded active pharmaceutical ingredient(s) is released over a period of from about 6 months to about 8 months, from about 6 months to about 10 months, from about 6 months to about 12 months, from about 6 months to about 14 months, from about 6 months to about 16 months, from about 6 months to about 18 months, from about 6 months to about 20 months, from about 6 months to about 22 months, from about 8 months to about 10 months, from about 8 months to about 12 months, from about 8 months to about 14 months, from about 8 months to about 16 months, from about 8 months to about 18 months, from about 8 months to about 20 months, from about 8 months to about 22 months, from about 8 months to about 24 months, from about 10 months to about 12 months, from about 10 months to about 14 months, from about 10 months to about 16 months, from about 10 months to about 18 months, from about 10 months to about 20 months, from about 10 months to about 22 months, from about 10 months to about 24 months, from about 12 months to about 14 months, from about 12 months to about 16 months, from about 12 months to about 18 months, from about 12 months to about 20 months, from about 12 months to about 22 months, from about 12 months to about 24 months, from about 14 months to about 16 months, from about 14 months to about 18 months, from about 14 months to about 20 months, from about 14 months to about 22 months, from about 14 months to about 24 months, from about 16 months to about 18 months, from about 16 months to about 20 months, from about 16 months to about 22 months, from about 16 months to about 24 months, from about 18 months to about 20 months, from about 18 months to about 22 months, from about 18 months to about 24 months, from about 20 months to about 22 months, from about 20 months to about 24 months, or from about 22 months to about 24 months.

In some aspects, the one or more free active pharmaceutical ingredient(s) is released over a period of about 1 month. In some aspects, the one or more free active pharmaceutical ingredient(s) is released over a period of about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months. In some aspects, the one or more free active pharmaceutical ingredient(s) is released over a period of about 6 months. In some aspects, the one or more free active pharmaceutical ingredient(s) is released over a period of about 12 months. In some aspects, the one or more free active pharmaceutical ingredient(s) is released over a period of about 18 months. In some aspects, the one or more free active pharmaceutical ingredient (s) is released over a period of about 24 months.

The present disclosure also provides compositions comprising a crosslinked hyaluronic acid and one or more free active pharmaceutical ingredients.

Certain crosslinked hyaluronic acids are known in the art, e.g., Juvederm® Ultra, Juvederm® Ultra Plus, Healaflow®, Restylane®, Puragen®, Hylaform®, and Captique®.

In some aspects, each of the one or more free active pharmaceutical ingredient(s) is independently selected from the group consisting of an intraocular pressure (IOP) lowering agent, a ciliary neurotrophic factor (“CNTF”) analog, a FAS inhibitor (also known as a FAS ligand inhibitor), a HIF-1 alpha inhibitor, a HIF-2 alpha inhibitor, a vascular endothelial growth factor (“VEGF”) inhibitor, a vasoactive intestinal peptide (“VIP”) analog, a histatin analog, an alpha crystallin aggregation inhibitor, a reducing agent, a mitochondrial stabilizer, and a tumor necrosis factor-alpha (“TNF-alpha”) inhibitor. Non-limiting examples of these agents are provided above.

In some aspects, the one or more free active pharmaceutical ingredient(s) is selected from the group consisting of bimatoprost, bimatoprost acid, brimonidine, netarsudil, latanoprost, tafluprost, travoprost, latanoprostene bunod, dorzolamide, brinzolamide, echothiophate iodide, unoprostone, dipivefrin, pilocarpine, metipranolol, omidenepag isopropyl, carteolol, carbachol, acetylcholine, apraclonidine, betaxolol, timolol, levobunolol, difluprednate, loteprednol, methylprednisolone acetate, triamcinolone acetate, betamethasone, triamcinolone hexacetonide, dexamethasone, fluocinolone, fluorometholone, triamcinolone, rimexolone, prednisolone, lifitegrast, cyclosporine, bromfenac, nepafenac, ketorolac, diclofenac, suprofen, flurbiprofen, moxifloxacin, gatifloxacin, ganciclovir, azithromycin, besifloxacin, tobramycin, trifluridine, vidarabine, erythromycin, levofloxacin, ofloxacin, gentamicin, chloramphenicol, natamycin, bacitracin, tropicamide, phenylephrine, homatropine, cyclopentolate, and atropine.

In some aspects, the individual concentrations of the one or more free active pharmaceutical ingredient(s) is from about 1 wt % to about 20 wt %. In some aspects, the individual concentrations of the one or more free active pharmaceutical ingredient(s) is from about 1 wt % to about 2 wt %, from about 1 wt % to about 4 wt %, from about 1 wt % to about 6 wt %, from about 1 wt % to about 8 wt %, from about 1 wt % to about 10 wt %, from about 1 wt % to about 12 wt %, from about 1 wt % to about 14 wt %, from about 1 wt % to about 16 wt %, from about 1 wt % to about 18 wt %, from about 2 wt % to about 4 wt %, from about 2 wt % to about 6 wt %, from about 2 wt % to about 8 wt %, from about 2 wt % to about 10 wt %, from about 2 wt % to about 12 wt %, from about 2 wt % to about 14 wt %, from about 2 wt % to about 16 wt %, from about 2 wt % to about 18 wt %, from about 2 wt % to about 20 wt %, from about 4 wt % to about 6 wt %, from about 4 wt % to about 8 wt %, from about 4 wt % to about 10 wt %, from about 4 wt % to about 12 wt %, from about 4 wt % to about 14 wt %, from about 4 wt % to about 16 wt %, from about 4 wt % to about 18 wt %, from about 4 wt % to about 20 wt %, from about 6 wt % to about 8 wt %, from about 6 wt % to about 10 wt %, from about 6 wt % to about 12 wt %, from about 6 wt % to about 14 wt %, from about 6 wt % to about 16 wt %, from about 6 wt % to about 18 wt %, from about 6 wt % to about 20 wt %, from about 8 wt % to about 10 wt %, from about 8 wt % to about 12 wt %, from about 8 wt % to about 14 wt %, from about 8 wt % to about 16 wt %, from about 8 wt % to about 18 wt %, from about 8 wt % to about 20 wt %, from about 10 wt % to about 12 wt %, from about 10 wt % to about 14 wt %, from about 10 wt % to about 16 wt %, from about 10 wt % to about 18 wt %, from about 10 wt % to about 20 wt %, from about 12 wt % to about 14 wt %, from about 12 wt % to about 16 wt %, from about 12 wt % to about 18 wt %, from about 12 wt % to about 20 wt %, from about 14 wt % to about 16 wt %, from about 14 wt % to about 18 wt %, from about 14 wt % to about 20 wt %, from about 16 wt % to about 18 wt %, from about 16 wt % to about 20 wt %, or from about 18 wt % to about 20 wt %. In any of the previous aspects, the total concentration of all the individual free active pharmaceutical ingredients is from about 1 wt % to about 50 wt %.

In some aspects, the concentration of each individual free active pharmaceutical ingredient is about 5 wt %. In some aspects, the concentration of each individual free active pharmaceutical ingredient is about 1 wt %, about 2 wt %, about 3 wt %, about 4 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt %.

In some aspects, the free active pharmaceutical ingredient is encapsulated, e.g., in a microsphere.

In some aspects, the composition further comprises a second polymer. In some aspects, the second polymer is a collagen or a poloxamer. In some aspects, the poloxamer is Pluronic® L64, Pluronic® P65, Pluronic® P84, Pluronic® P85, Pluronic® F88, Pluronic® P103, Pluronic® P104, Pluronic® P105, Pluronic® F108, Pluronic® F123, or Pluronic® F127. In some aspects, the collagen is VitriCol®.

In some aspects, the concentration of the second polymer is from about 1 mg/mL to about 250 mg/mL. In some aspects, the concentration of the second polymer is from about 1 mg/mL to about 5 mg/mL. In some aspects, the concentration of the second polymer is from about 1 mg/mL to about 10 mg/mL, from about 1 mg/mL to about 50 mg/mL, from about 1 mg/mL to about 100 mg/mL, from about 1 mg/mL to about 150 mg/mL, from about 1 mg/mL to about 200 mg/mL, from about 5 mg/mL to about 10 mg/mL, from about 5 mg/mL to about 50 mg/mL, from about 5 mg/mL to about 100 mg/mL, from about 5 mg/mL to about 150 mg/mL, from about 5 mg/mL to about 200 mg/mL, from about 5 mg/mL to about 250 mg/mL, from about 10 mg/mL to about 50 mg/mL, from about 10 mg/mL to about 100 mg/mL, from about 10 mg/mL to about 150 mg/mL, from about 10 mg/mL to about 200 mg/mL, from about 10 mg/mL to about 250 mg/mL, from about 50 mg/mL to about 100 mg/mL, from about 50 mg/mL to about 150 mg/mL, from about 50 mg/mL to about 200 mg/mL, from about 50 mg/mL to about 250 mg/mL, from about 100 mg/mL to about 150 mg/mL, from about 100 mg/mL to about 200 mg/mL, from about 100 mg/mL to about 250 mg/mL, from about 150 mg/mL to about 200 mg/mL, from about 150 mg/mL to about 250 mg/mL, or from about 200 mg/mL to about 250 mg/mL.

In some aspects, the concentration of the second polymer is about 5 mg/mL. In some aspects, the concentration of the second polymer is about 1 mg/mL, about 10 mg/mL, about 25 mg/mL, about 50 mg/mL, about 75 mg/mL, about 100 mg/mL, about 125 mg/mL, about 150 mg/mL, about 175 mg/mL, about 200 mg/mL, about 225 mg/mL, or about 250 mg/mL.

In some aspects, the concentration of the second polymer is from about 0.1 wt % to about 25 wt %. In some aspects, the concentration of the second polymer is from about 0.1 wt % to about 0.5 wt %. In some aspects, the concentration of the second polymer is from about 0.1 wt % to about 1 wt %, from about 0.1 wt % to about 5 wt %, from about 0.1 wt % to about 10 wt %, from about 0.1 wt % to about 15 wt %, from about 0.1 wt % to about 20 wt %, from about 0.5 wt % to about 1 wt %, from about 0.5 wt % to about 5 wt %, from about 0.5 wt % to about 10 wt %, from about 0.5 wt % to about 15 wt %, from about 0.5 wt % to about 20 wt %, from about 0.5 wt % to about 25 wt %, from about 1 wt % to about 5 wt %, from about 1 wt % to about 10 wt %, from about 1 wt % to about 15 wt %, from about 1 wt % to about 20 wt %, from about 1 wt % to about 25 wt %, from about 5 wt % to about 10 wt %, from about 5 wt % to about 15 wt %, from about 5 wt % to about 20 wt %, from about 5 wt % to about 25 wt %, from about 10 wt % to about 15 wt %, from about 10 wt % to about 20 wt %, from about 10 wt % to about 25 wt %, from about 15 wt % to about 20 wt %, from about 15 wt % to about 25 wt %, or from about 20 wt % to about 25 wt %.

In some aspects, the concentration of the second polymer is about 0.5 wt %. In some aspects, the concentration of the second polymer is about 0.1 wt %, about 1 wt %, about 2.5 wt %, about 5 wt %, about 7.5 wt %, about 10 wt %, about 12.5 wt %, about 15 wt %, about 17.5 wt %, about 20 wt %, about 22.5 wt %, or about 25 wt %.

In some aspects, the one or more free active pharmaceutical ingredient(s) is released over a period of from about 6 months to about 24 months. In some aspects, the one or more free active pharmaceutical ingredient(s) is released over a period of from about 6 months to about 8 months, from about 6 months to about 10 months, from about 6 months to about 12 months, from about 6 months to about 14 months, from about 6 months to about 16 months, from about 6 months to about 18 months, from about 6 months to about 20 months, from about 6 months to about 22 months, from about 8 months to about 10 months, from about 8 months to about 12 months, from about 8 months to about 14 months, from about 8 months to about 16 months, from about 8 months to about 18 months, from about 8 months to about 20 months, from about 8 months to about 22 months, from about 8 months to about 24 months, from about 10 months to about 12 months, from about 10 months to about 14 months, from about 10 months to about 16 months, from about 10 months to about 18 months, from about 10 months to about 20 months, from about 10 months to about 22 months, from about 10 months to about 24 months, from about 12 months to about 14 months, from about 12 months to about 16 months, from about 12 months to about 18 months, from about 12 months to about 20 months, from about 12 months to about 22 months, from about 12 months to about 24 months, from about 14 months to about 16 months, from about 14 months to about 18 months, from about 14 months to about 20 months, from about 14 months to about 22 months, from about 14 months to about 24 months, from about 16 months to about 18 months, from about 16 months to about 20 months, from about 16 months to about 22 months, from about 16 months to about 24 months, from about 18 months to about 20 months, from about 18 months to about 22 months, from about 18 months to about 24 months, from about 20 months to about 22 months, from about 20 months to about 24 months, or from about 22 months to about 24 months.

In some aspects, the one or more free active pharmaceutical ingredient(s) is released over a period of about 1 month. In some aspects, the one or more free active pharmaceutical ingredient(s) is released over a period of about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months. In some aspects, the one or more free active pharmaceutical ingredient(s) is released over a period of about 6 months. In some aspects, the one or more free active pharmaceutical ingredient(s) is released over a period of about 12 months. In some aspects, the one or more free active pharmaceutical ingredient(s) is released over a period of about 18 months. In some aspects, the one or more free active pharmaceutical ingredient (s) is released over a period of about 24 months.

In some aspects, the composition comprises crosslinked hyaluronic acid, collagen, and at least one free active pharmaceutical ingredient.

In some aspects, the composition comprises crosslinked hyaluronic acid, collagen, and bimatoprost acid.

In some aspects, the composition comprises from about 4 mg/mL to about 12 mg/mL crosslinked hyaluronic acid, from about 0.5 mg/mL to about 4 mg/mL collagen, and from about 1 mg/mL to about 10 mg/mL of at least one free active pharmaceutical ingredient.

In some aspects, the composition comprises from about 4 mg/mL to about 12 mg/mL crosslinked hyaluronic acid, from about 0.5 mg/mL to about 4 mg/mL collagen, and from about 1 mg/mL to about 10 mg/mL bimatoprost acid.

In some aspects, the composition comprises crosslinked hyaluronic acid, a histatin analog, a CNTF analog, and, optionally a VIP analog.

In some aspects, the composition comprises crosslinked hyaluronic acid and a CNTF analog and/or a FAS inhibitor and/or a prostaglandin analog.

In some aspects, the composition comprises crosslinked hyaluronic acid and a CNTF analog and/or a FAS inhibitor and, optionally, a VEGF inhibitor.

In some aspects, the composition comprises crosslinked hyaluronic acid and a CNTF analog and/or a FAS inhibitor.

### Methods of Use

The present disclosure also provides methods of delivering one or more active pharmaceutical ingredient(s) to the anterior chamber, posterior chamber, suprachoroidal space, or vitreous humor of an eye of a subject in need thereof, the method comprising injecting a composition disclosed herein into the eye of the subject.

The present disclosure also provides methods of treating glaucoma, age-related macular degeneration (AMD), macular degeneration (MD), uveitis, retinal vein occlusion, macular edema, macular telangiectasia, endophthalmitis, retinitis, dysfunctional lens syndrome (DLS), Fuch's Dystrophy, Sjögren's Syndrome, Stargardt disease, cataract, diabetic retinopathy, ocular hypertension, optic neuritis, corneal dystrophies, keratitis, scleritis, amblyopia, strabismus, blepharitis, blepharospasm, central retinal vein occlusion, coloboma, ocular histoplasmosis syndrome, conjunctivitis, presbyopia, or dry eye in a subject in need thereof, the method comprising administering the hyaluronic acid disclosed herein or a composition disclosed herein to the eye of the subject.

In some aspects, the composition is administered to the anterior chamber, posterior chamber, suprachoroidal space, or vitreous humor of the eye of the subject.

In some aspects, the glaucoma is open-angle glaucoma, angle-closure glaucoma, congenital glaucoma, or secondary glaucoma.

The present disclosure also provides methods of delivering one or more active pharmaceutical ingredient(s) to a joint of a subject in need thereof, the method comprising injecting the hyaluronic acid disclosed herein or a composition disclosed herein into the joint of the subject.

The present disclosure also provides methods of treating osteoarthritis in a subject in need thereof, the method comprising administering the hyaluronic acid disclosed herein or a composition disclosed herein to the joint of the subject.

The present disclosure also provides methods of delivering one or more active pharmaceutical ingredient(s) to a tooth, root, or gum of a subject in need thereof, the method comprising injecting the hyaluronic acid disclosed herein or a composition disclosed herein into the tooth, root, or gum of the subject.

The present disclosure also provides methods of treating a dental or periodontal disease, disorder, or condition in a subject in need thereof, the method comprising administering the hyaluronic acid disclosed herein or a composition disclosed herein to the tooth, root, or gum of the subject.

The disclosure also provides the sequences listed in Table 2.

TABLE 2

| SEQ | | |
|---|---|---|
| ID NO. | Sequence | Description |
| 1 | Ac-VGDGGLFEKKL-NH₂ | Peptide 6 |
| 2 | Ac-DGGL^{A}G-NH₂ | Peptide 21 |
| 3 | HHIYLGAVNYIY | MET12 |
| 4 | HHIYLGATNYIY | MET12 |
| | | derivative |
| 5 | YLGA | Compound 1 |
| 6 | IYLGA | Compound 2 |
| 7 | YLGAV | Compound 3 |
| 8 | HIYLGA | Compound 4 |
| 9 | IYLGAV | Compound 5 |
| 10 | HIYLGAV | Compound 6 |
| 11 | IYLGAVN | Compound 7 |
| 12 | HHIYLGA | Compound 8 |
| 13 | YLGAVNY | Compound 9 |
| 14 | HHIYLGAV | Compound 10 |
| 15 | YLGAVNYI | Compound 11 |
| 16 | 5′-GAAACGAACUGCACCCGGAU-3′ | FAS siRNA |
| | | sense |
| 17 | 5′-UAGCGACUAAACACAUCAA-3′ | negative |
| | | siRNA sense |
| 18 | DGGL | Neurotrophic |
| | | peptide |
| 19 | YLGAVN | MET12 |
| | | derivative |
| 20 | HIYLGAVN | MET12 |
| | | derivative |

The following examples are illustrative and do not limit the scope of the claimed aspects.

### EXAMPLES

### Example 1

### Amide Based HA Conjugates

Sodium hyaluronate is converted to the tert-butyl ammonium (TBA) salt. Sodium HA with a molecular weight (M.W.) of 500K Da to 2M Da is converted to the TBA salt by passing through an Amberlite IR-120 ion exchange resin in TBA form. The eluate is then vacuum dried or lyophilized for future use. To synthesize the amide based hydrophobic conjugates, 1 gram of HA TBA is dissolved in 100 mL of DMSO. Catalytic amounts of methane sulfonic acid and 1,1′-carbonyldiimidazole are added and activated by stirring for 60 minutes at room temperature. After 60 minutes an alkylamine is added and stirred overnight at 42° C. to yield the final product.

### Example 2

### Ester Based HA Conjugates

Sodium hyaluronate is converted to the tert-butyl ammonium (TBA) salt. To synthesize the ester based hydrophobic conjugates 1 gram of HA TBA is dissolved in 100 mL of DMSO. The appropriate amount of alkyl halide is added with stirring at 30° C. for 12 hours to form the ester.

### Example 3

### Lysine Based HA Conjugates

Sodium hyaluronate is converted to the tert-butyl ammonium (TBA) salt. HA is conjugated with lysine using EDC/NHS activation of the HA carboxylic acid. HA TBA is dissolved in DMSO with EDC (1-Ethyl-3-[3-dimethylaminopropyl]carbodiimide hydrochloride) then NHS (N-hydroxysuccinimide) to form an amine reactive NHS ester of HA. The reactive NHS HA ester is then added to a solution of lysine to create the lysine amide of HA.

The HA-lysine amide is reacted with 1,4-butanediol which can then be used to esterify the C-terminus of peptides or carboxylic acid residues of small molecule active pharmaceutical ingredients such as bimatoprost.

Alternatively, the HA-lysine amide is reacted with bromobutyrate which can then be used to esterify the C-terminus of peptides or carboxylic acid residues of small molecule active pharmaceutical ingredients such as bimatoprost.

### Example 4

### Direct Ester HA Conjugates

Sodium hyaluronate is converted to the tert-butyl ammonium (TBA) salt. To synthesize the ester based hydrophobic conjugates 1 gram of HA TBA is dissolved in 100 mL of DMSO. The appropriate amount of alkyl halide, e.g., an alkyl halide linked to an active pharmaceutical ingredient, is added with stirring at 30° C. for 12 hours to form the ester.

### Example 5

### Disulfide Based Cleavable HA Conjugates

HA-TBA salt is dissolved in N,N-dimethylformamide (DMF) with 2% LiCl at 90° C. until in solution then cooled to room temperature. Pyridine is added with chloroacetyl chloride and reacted for 4 hours to form the chloroacetyl modified HA. The chloroacetyl HA is then reacted with excess dithiothreitol at pH 7.4 in an aqueous buffer. The thiolated HA thus formed can be used to form disulfide prodrugs of sulfhydryl contain compounds such as cysteine containing peptides. Substituents can be added to the dithiothreitol to control the oxidative cleavage rate.

### Example 6

### Intravitrial Injection of Bimatoprost Depot in Beagle Dogs

The efficacy and tolerability of an intravitreal injection of bimatoprost acid (BA) microspheres in a crosslinked hyaluronic acid (HA) and collagen depot was assessed in beagle dogs. A sustained-release formulation containing 5.5 mg/mL bimatoprost acid and 0.8% hyaluronic acid and 0.2% collagen, as well as a placebo formulation, were administered. The test articles are shown in Table 3.

TABLE 3

| Ingredient | Concentration (active) | Concentration (Placebo) |
|---|---|---|
| Bimatoprost acid | 5.5 mg/mL | 0 mg/mL |
| Crosslinked HA | 8 mg/mL | 8 mg/mL |
| Human collagen | 2 mg/mL | 2 mg/mL |
| Phosphate buffered | q.s. to volume | q.s. to volume |
| saline (PBS) | | |

The test article was manufactured in a biologic safety cabinet to ensure sterility. A 4 mg/mL human collagen solution (VitriCol®) was prepared in water for injection (WIFI) at pH 2.0. The pH of the WIFI was adjusted to 2.0 with HCL. Crosslinked HA (Juvederm® Ultra, 24 mg/mL HA) was used as received. A 0.5 mL volume of the collagen solution and a 0.333 mL volume of the HA gel were added to a 1-mL syringe. 100 μL of a 10×PBS solution was added to the same syringe. The 1-mL syringe was connected to a 3-mL syringe through a rapid fill connector. The mixture was made homogeneous by pushing the material through the rapid fill connector into the 3-mL syringe and back into the 1-mL syringe 50 times. The final gel was pushed back into the 1-mL syringe.

Bimatoprost acid containing microspheres were then incorporated into the gel. 220 mg of BA microspheres were added to a separate 3-mL syringe. This syringe was connected to the 1-mL syringe of gel via a rapid fill connector. The microspheres and gel were then mixed by passing through the connector 50 times, leaving the final product in the 1-mL syringe.

The vehicle control was manufactured using the same process and formulation as the active formulation, simply leaving out the BA microspheres.

Beagle dogs were used to assess the efficacy and tolerability of the BA-HA gel. Following an initial tonometry acclimation phase, animals received a single intravitreal injection of placebo article (OS) and test article (OD) on Day 1. Ocular examinations, tonometry, pupillometry, fundus imaging, fluorescein angiography, slit lamp images and OCT imaging were performed over 84 days. Histology was conducted on day 84.

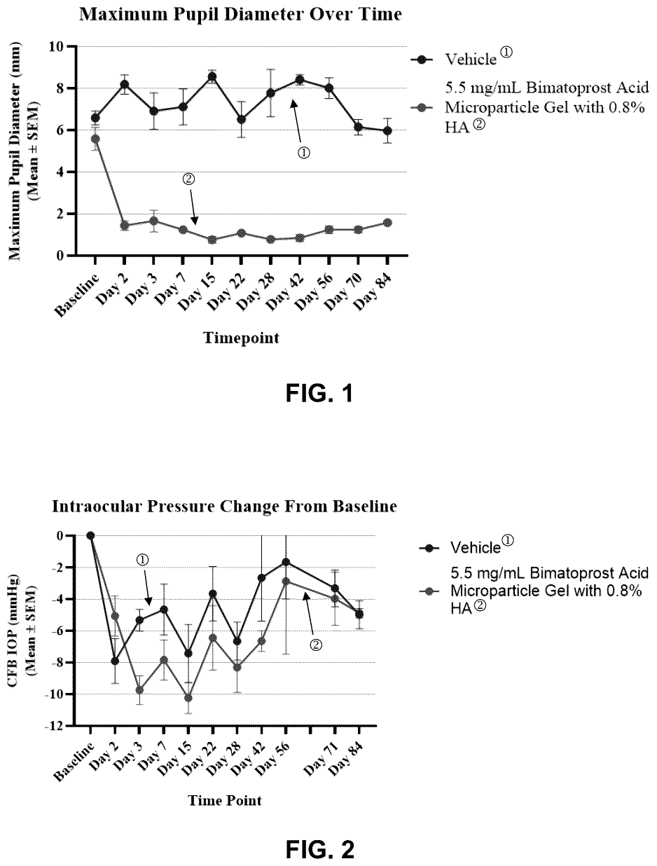

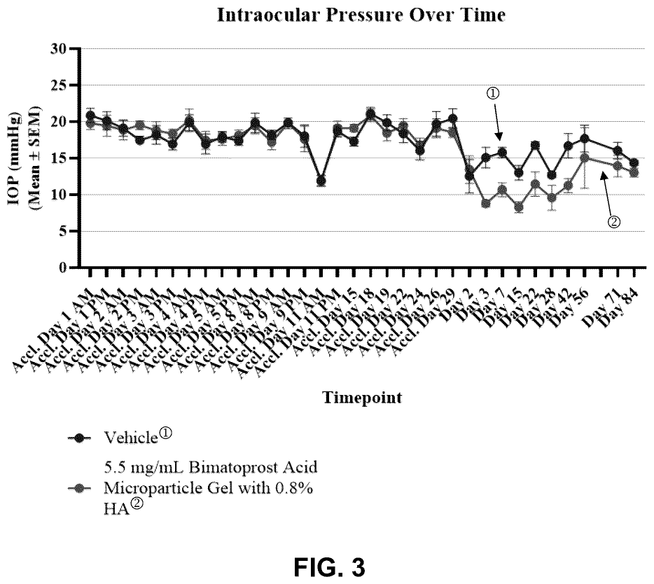

A 50% drop in IOP over the initial baseline was observed with the BA sustained release formulation after a single injection over the first months followed by a 25% drop over the next 84 days, as shown in FIGS. 2 and 3. Significant miosis in the treated eye was observed over the 84 days demonstrating efficacy, as shown in FIG. 1. Prostaglandins such as bimatoprost acid cause significant miosis in dogs. The formulation was well tolerated by all measures in three of the dogs over the course of the study. One animal exhibited inflammation in the treated eye by day 21.

Non-crosslinked HA is known to clear from the posterior segment fast relative to a desired 3 to 6 month delivery profile. The vitreal half-life of HA depends on its molecular weight and degree of crosslinking. Uncrosslinked HA has a vitreal half-life of weeks, though some estimates are as short as 2 days (Nakagawa M, Tanaka M, Miyata T. Evaluation of collagen gel and hyaluronic acid as vitreous substitutes. Ophthalmic Res. 1997; 29(6):409-20. doi: 10.1159/000268042. PMID: 9380343). HA is cleared from the vitreous by dissolution and elimination out of the anterior chamber or by metabolism by hyaluronidases. Crosslinking and crosslinking density can modify clearance and extend duration.

This example demonstrates the utility of the crosslinked-HA depot. The duration of the crosslinked-HA depot in the vitreous exceeded 84 days. Both miosis and IOP reduction from the bimatoprost was observed over the 84 days indicating the presence of the depot. Moreover, the sequestered microparticles in the depot did not migrate en masse throughout the eye and into the anterior chamber. Anterior chamber material was not observed over 56 days post injection in the three eyes receiving with the crosslinked-HA bimatoprost depot and over the full 84 days with 2 eyes. One eye demonstrated some material in the anterior chamber beginning at day 70. This, however, was observed with the vehicle control that did not contain microspheres, indicating that it was not migration of the microparticles in that eye.

### Example 7

### Suprachoroidal Injection of Bimatoprost Depot in Beagle Dogs

Bimatoprost acid (BA) microspheres suspended in hyaluronic acid were dosed by suprachoroidal injection to beagle dogs and assessed for intraocular pressure reduction (IOP) and tolerability. Each of 3 dogs received a 100 μL suprachoroidal dose OD of the BA formulation, as well as 100 μL of vehicle article OS. The active test article contained 6.12 mg/mL of BA delivered in a microsphere formulation suspended in HA (Healon® Pro). The test article was manufactured by placing 241.2 mg of BA microspheres in a 1-mL syringe. To the same syringe 0.85 mL of HA was added through a rapid fill connector. The 1-mL syringe was then connected to a 3-mL syringe through a rapid fill connector. The mixture was made homogeneous by pushing the material through the rapid fill connector into the 3-mL syringe and back into the 1-mL syringe 50 times. The final gel was pushed back into the 1 mL syringe.

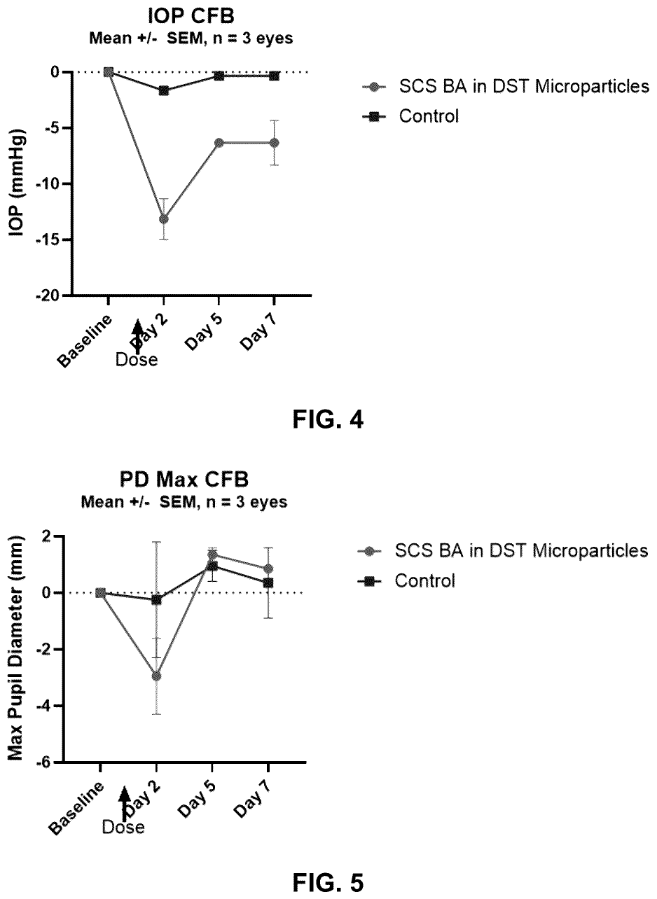

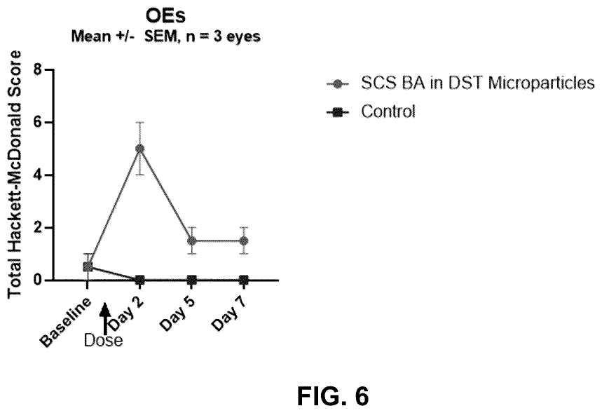

The BA formulation was administered to the beagle dogs by injecting 100 μL from the 1-mL syringe through a 27 gauge needle into the suprachoroidal space. Ocular examinations, IOP/pupillometry, and fundus imaging were taken on days 2, 5, and 7. Posterior segment OCT was conducted on day 7. The IOP reduced by 60% at day 2 and 29% at days 5 sand 7 in the active BA group. The formulations were also well tolerated over the 7-day study. The change from baseline (CFB) for IOP and pupil diameter are shown in FIGS. 4 and 5, respectively. The Hackett McDonald tolerability scores are shown in FIG. 6.

What is claimed is:

1. Hyaluronic acid comprising a compound of Formula (I): 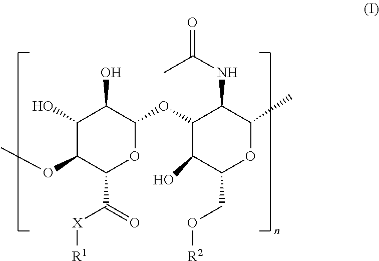
or a salt or solvate thereof, wherein: n is an integer from 500 to 25,000;
X at each occurrence is selected from the group consisting of —NH— and —O—;
R¹ and R² at each occurrence are independently selected from the group consisting of hydrogen, a hydrophobic group, -L^{J}-L²-A, and -A; each L¹ is an amino acid or absent; each L² is a linker; and
each A is independently a covalently bonded active pharmaceutical ingredient, wherein each instance of -A can be the same or different.

2. The hyaluronic acid of claim 1, wherein n is from 1,200 to 5,000.

3. The hyaluronic acid of claim 1, wherein the hydrophobic group is selected from the group consisting of optionally substituted Ci-Cis alkyl and an amino acid.

4. The hyaluronic acid of claim 3, wherein the hydrophobic group is a Ci-Cis alkyl that is selected from the group consisting of:

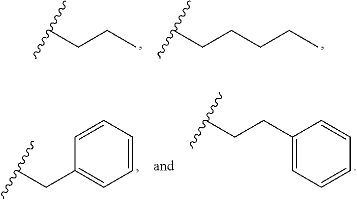

5. The hyaluronic acid of claim 3, wherein the amino acid is selected from the group consisting of:

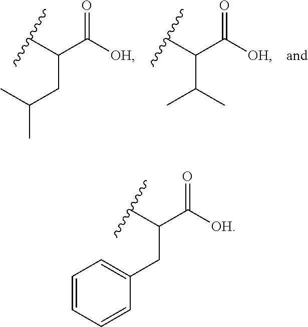

6. The hyaluronic acid of claim 1, wherein L¹ is selected from the group consisting of

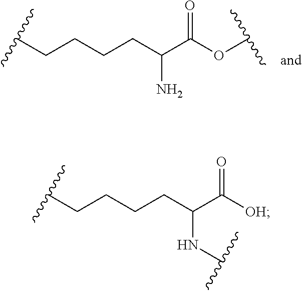 [SMILES: *CCCCC(N)C(=O)O* ; *CCCCC(N*)C(=O)O]

7. The hyaluronic acid of claim 1, wherein L² is selected from the group consisting of:

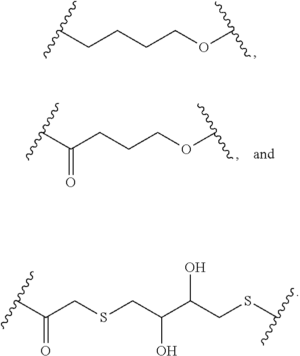 [SMILES: *CCCCO* ; *OCCCC(*)=O ; *SCC(O)C(O)CSCC(*)=O]

8. The hyaluronic acid of claim 1, wherein each A is independently selected from the group consisting of an intraocular pressure (IOP) lowering agent, a CNTF analog, a FAS inhibitor or FAS ligand inhibitor, a HIF-1 alpha inhibitor, a HIF-2 alpha inhibitor, a VEGF inhibitor, a VIP analog, a histatin analog, an alpha crystallin aggregation inhibitor, a reducing agent, a mitochondrial stabilizer, and a TNF-alpha inhibitor.

9. The hyaluronic acid of claim 8, wherein the IOP lowering agent is bimatoprost, bimatoprost acid, travoprost, travoprost acid, latanoprost, latanoprost acid, latanoprostene, tafluprost, or tafluprost acid; the CNTF analog is SEQ ID NO: 18, SEQ ID NO.: 1, SEQ ID NO: 2, or recombinant CNTF; or the FAS inhibitor is bicyclol, FLIP, MET12 (SEQ ID NO: 3), compound 1 (SEQ ID NO: 5), compound 2 (SEQ ID NO: 6), compound 3 (SEQ ID NO: 7), compound 4 (SEQ ID NO: 8), compound 5 (SEQ ID NO: 9), compound 6 (SEQ ID NO: 10), compound 7 (SEQ ID NO: 11), compound 8 (SEQ ID NO: 12), compound 9 (SEQ ID NO: 13), compound 10 (SEQ ID NO: 14), compound 11 (SEQ ID NO: 15), ONL1204, SEQ ID NO: 4, FAIM, NOL3, human decoy receptor 1 (DcRl), human decoy receptor 2 (DcR2), or human decoy receptor 3 (DcR3).

10. The hyaluronic acid of claim 1, wherein from about 1% to about 50% of the R groups are hydrophobic groups.

11. The hyaluronic acid of claim 1, wherein from about 1% to about 50% of the R groups are -L^{J}-L²-A or -A.

12. The hyaluronic acid of claim 1, wherein each -A of Formula (I) is selected from one active pharmaceutical ingredient, from two active pharmaceutical ingredients, or from three active pharmaceutical ingredients.

13. The hyaluronic acid of claim 1, comprising a first compound of Formula (I) that is crosslinked with a second compound of Formula (I), wherein the first compound comprises a first active pharmaceutical ingredient and the second compound comprises a second active pharmaceutical ingredient.

14. The hyaluronic acid of claim 1, comprising a first compound of Formula (I) that is crosslinked with a second compound of Formula (I) and a third compound of Formula (I), wherein the first compound comprises a first active pharmaceutical ingredient, the second compound comprises a second active pharmaceutical ingredient, and the third compound comprises a third active pharmaceutical ingredient.

15. A composition comprising the hyaluronic acid of claim 1 and a free active pharmaceutical ingredient.

16. The composition of claim 15, wherein the free active pharmaceutical ingredient is an IOP lowering agent that is bimatoprost, bimatoprost acid, travoprost, travoprost acid, latanoprost, latanoprost acid, latanoprostene, tafluprost, or tafluprost acid.

17. The composition of claim 15, wherein the hyaluronic acid comprises a first compound of Formula (I) that is crosslinked with a second compound of Formula (I), wherein the first compound comprises a first active pharmaceutical ingredient and the second compound comprises a second active pharmaceutical ingredient.

18. The composition of claim 17, wherein the first active pharmaceutical ingredient is a CNTF analog and the second active pharmaceutical ingredient is a FAS inhibitor.

19. The hyaluronic acid of claim 14, wherein the first active pharmaceutical ingredient is a CNTF analog, the second active pharmaceutical ingredient is a FAS inhibitor, and the third active pharmaceutical agent is a prostaglandin analog.

20. A method of delivering an active pharmaceutical ingredient to an eye of a subject in need thereof, the method comprising injecting the hyaluronic acid of claim 1 into the eye of the subject.

21. The method of claim 20, wherein the method further comprises injecting a free active pharmaceutical agent into the eye with the hyaluronic acid.

22. The method of claim 20, wherein the active pharmaceutical ingredient is delivered to the anterior chamber, posterior chamber, suprachoroidal space, or vitreous humor of the eye.

23. A method of treating an ophthalmic disease, disorder, or condition in a subject in need thereof, the method comprising administering the hyaluronic acid of claim 1 into an eye of the subject.

24. The method of claim 23, wherein the method further comprises administering a free active pharmaceutical agent into the eye with the hyaluronic acid.

25. The method of claim 23, wherein the ophthalmic disease, disorder, or condition is age-related macular degeneration (AMD), macular degeneration (MD), uveitis, retinal vein occlusion, macular edema, macular telangiectasia, endophthalmitis, retinitis, dysfunctional lens syndrome (DLS), Fuch's Dystrophy, Sjogren's Syndrome, Stargardt disease, cataract, diabetic retinopathy, ocular hypertension, optic neuritis, corneal dystrophies, keratitis, scleritis, amblyopia, strabismus, blepharitis, blepharospasm, central retinal vein occlusion, coloboma, ocular histoplasmosis syndrome, conjunctivitis, presbyopia, or dry eye.

26. The method of claim 23, wherein the composition is administered to the anterior chamber, posterior chamber, suprachoroidal space, or vitreous humor of one or both eyes of the subject.


SEQUENCE LISTING (20 sequences)

SEQ ID NO: 1 - protein, 11 aa, synthetic construct
  source 1..11: note = Peptide 6
  MOD_RES 1: note = Acetylation
  MOD_RES 11: note = Amidation
VGDGGLFEKKL

SEQ ID NO: 2 - protein, 5 aa, synthetic construct
  source 1..5: note = Peptide 21
  MOD_RES 1: note = Acetylation
  MOD_RES 5: note = Amidation
DGGLG

SEQ ID NO: 3 - protein, 12 aa, synthetic construct
  source 1..12: note = MET12
HHIYLGAVNYIY

SEQ ID NO: 4 - protein, 12 aa, synthetic construct
  source 1..12: note = MET12 derivative
HHIYLGATNYIY

SEQ ID NO: 5 - protein, 4 aa, synthetic construct
  source 1..4: note = Compound 1
YLGA

SEQ ID NO: 6 - protein, 5 aa, synthetic construct
  source 1..5: note = Compound 2
IYLGA

SEQ ID NO: 7 - protein, 5 aa, synthetic construct
  source 1..5: note = Compound 3
YLGAV

SEQ ID NO: 8 - protein, 6 aa, synthetic construct
  source 1..6: note = Compound 4
HIYLGA

SEQ ID NO: 9 - protein, 6 aa, synthetic construct
  source 1..6: note = Compound 5
IYLGAV

SEQ ID NO: 10 - protein, 7 aa, synthetic construct
  source 1..7: note = Compound 6
HIYLGAV

SEQ ID NO: 11 - protein, 7 aa, synthetic construct
  source 1..7: note = Compound 7
IYLGAVN

SEQ ID NO: 12 - protein, 7 aa, synthetic construct
  source 1..7: note = Compound 8
HHIYLGA

SEQ ID NO: 13 - protein, 7 aa, synthetic construct
  source 1..7: note = Compound 9
YLGAVNY

SEQ ID NO: 14 - protein, 8 aa, synthetic construct
  source 1..8: note = Compound 10
HHIYLGAV

SEQ ID NO: 15 - protein, 8 aa, synthetic construct
  source 1..8: note = Compound 11
YLGAVNYI

SEQ ID NO: 16 - RNA, 20 nt, synthetic construct
  source 1..20: mol_type = other RNA; note = FAS siRNA sense
gaaacgaactgcacccggat

SEQ ID NO: 17 - RNA, 19 nt, synthetic construct
  source 1..19: mol_type = other RNA; note = negative siRNA sense
tagcgactaaacacatcaa

SEQ ID NO: 18 - protein, 4 aa, synthetic construct
  source 1..4: note = Neurotrophic peptide
DGGL

SEQ ID NO: 19 - protein, 6 aa, synthetic construct
  source 1..6: note = MET12 derivative
YLGAVN

SEQ ID NO: 20 - protein, 8 aa, synthetic construct
  source 1..8: note = MET12 derivative
HIYLGAVN